(19) United States
(12) Patent Application Publication
(10) Pub. No.: US 2012/0149663 A1
(43) Pub. Date: Jun. 14, 2012

(54) BORONIC ACID COMPOSITIONS AND METHODS RELATED TO CANCER

(75) Inventors: Milton L. Brown, Brookeville, MD (US); Venkata M. Yenugonda, McLean, VA (US); Yali Kong, Centerville, VA (US)

(73) Assignee: GEORGETOWN UNIVERSITY, Washington, DC (US)

(21) Appl. No.: 13/390,731

(22) PCT Filed: Aug. 18, 2010
(86) PCT No.: PCT/US10/45925

§ 371 Date: Feb. 16, 2012

Related U.S. Application Data
(60) Provisional application No. 61/234,991, filed on Aug. 18, 2009.

(51) Int. Cl. A61K 31/69; A61P 35/00; C07F 5/02

(52) U.S. Cl. 514/64; 548/110; 564/8; 568/3; 568/6

(57) ABSTRACT

Disclosed are compounds and methods related to boronic acid derivatives of resveratrol. Certain of these derivatives have enhanced efficacy relative to resveratrol, function as irreversible modulators, and act at the GI/S phase of the cell cycle.

### CROSS-REFERENCE TO RELATED APPLICATIONS

This application claims benefit of U.S. Provisional Application No. 61/234,991, filed Aug. 18, 2009, and is hereby incorporated by reference in its entirety.

### SUMMARY

The objects, advantages and features of the compounds, compositions and methods disclosed herein will become more apparent when reference is made to the following description taken in conjunction with the accompanying drawings.

Disclosed herein are compounds, compositions and methods. The compounds and methods are related to boronic acid derivatives of resveratrol.

In some forms, the compounds, compositions and methods relate to the formula A-L-C, wherein pharmaceutically acceptable salt, prodrug, clathrate, tautomer or solvate thereof comprising, compounds of structure A-L-C, or a pharmaceutically acceptable salt, prodrug, clathrate, tautomer or solvate thereof, wherein:

A is substituted or unsubstituted cylcoalkyl, aryl, heteroaryl, heterocyclyl;

L is present or absent, if present L is a linker; and

C is substituted or unsubstituted cylcoalkyl, aryl, heteroaryl, heterocyclyl, wherein at least one position in the compound is substituted with —B(OH)₂, and at least one position in the compound is substituted with alkoxy, alkoxydialkylamino or hydroxyl.

In some forms, the compound, compositions and methods relate to treatment of cancer.

### BACKGROUND

Breast cancer is one of the most common types of cancer and a major cause of death among women in the United States. Estrogen receptors (ERα and ERβ) play an important role in the development of many breast tumor cells through binding of 17b-estradiol and stimulate the transcriptional genes in developing breast cancer. (Service RF., Science 1998; 279(5357):1631-1633, Sommer S, et al., Semin Cancer Biol 2001; 11(5):339-352.) One approach to inhibiting estrogen-responsive genes is to block the receptors with antagonists from natural or semi-synthetic origin.

Resveratrol (RSV) is a natural compound found in the skin of red grapes and other food products that seems to have a wide spectrum of biological activities which includes phytoalexin to protect plants against the fungal infection (Plant Mol Biol, 15: 325-335, 1990), as a cardioprotective agent (Nutr Res, 28: 729-737, 2008), partially preventing platelet aggregation (Clin Chim. Acta, 246: 163-182, 1996; J Nat Prod, 60:1082-1087, 1997), and inhibiting 5-lipoxygenase activity and prostaglandin synthesis (Mol Pharmacol 54: 445-451, 1998; Biochem Pharmacol 59, 865-870, 2000). Use of resveratrol in the pharmaceutical and cosmetic fields was previously disclosed (WO9959561; WO9958119; EP0773020; FR2766176; WO9904747).

There is an interest in resveratrol as a chemo preventive agent in cancer therapy based on its striking inhibitory effects on cellular events associated with cancer initiation, promotion and propagation (Science, 275, 218; 1997; J Nutr Biochem, 16: 449, 2005; Cancer Lett, 269, 243:2008). Previous studies on in vitro anti cancer effects of resveratrol showed that it interacts with multiple molecular targets and has positive effects on different cancer cells including breast, skin, gastric, colon, prostate, leukemia. (Nat. Rev. Drug Discov, 5, 493: 2006) However, the study of pharmacokinetics of resveratrol in humans concluded that even high doses of resveratrol might be insufficient to achieve resveratrol concentrations required for the systemic prevention of cancer (Toxicol. Appl. Pharmacol., 224: 274, 2007) because of its lower bioavailability and its quick metabolization as sulfo and glucuro conjugates. (Cancer Epidemiol. Biomarkers Prev, 16: 1246, 2007, J. Nutr. 136: 2542, 2006 Drug Metab Dispos. 32: 1377, 2004 Mol. Nutr. Food Res, 49: 482, 2005). Other studies have focused on the design and synthesis of novel resveratrol analogs with more potent antitumor activity and a better pharmacokinetic profile. (J Med Chem, 46:3546, 2003 Med Chem, 48: 1292, 2005, J Med Chem, 48: 6783, 2005 Cancer Chemother. Pharmacol, 63: 27, 2008, J Med Chem 49, 7182, 2006 J. Agric. Food Chem. 58, 226, 2010).

There are previous reports on a boronic acid biostere of combrestatin A-4 and a chalcone analog of combrestatin A-4 as potent anti cancer agents (Chem Biol, 12: 1007, 2005, Bioorg. Med. Chem., 18, 971, 2010) In addition boronic acid and ester compounds have been reported to display a variety of pharmaceutically useful biological activities as proteosome inhibitors and several important functions including reduction in the rate of muscle protein degradation, reduction in the activity of NF-kB in a cell, inhibition in the cyclin degradation in a cell, inhibition in the growth of cancer cells, and inhibition of antigen presentation in a cell (Cell, 79: 13-21, 1991; Cancer Res, 70: 1970-80, 2010, Bioorg Med Chem Lett, 10: 3416-9, 2010 J. Med. Chem., 51: 1068-1072, 2008, U.S. Pat. No. 4,499,082, 1985, U.S. Pat. No. 5,187,157,1993, U.S. Pat. No. 5,242,904, 1993, U.S. Pat. No. 5,250,720, 1993, U.S. Pat. No. 5,169,841, 1992, U.S. Pat. No. 5,780,454 1998, U.S. Pat. No. 6,066,730, 2000, U.S. Pat. No. 6,083,903, 2000, U.S. Pat. No. 6,297,217, 2001. These unique structural features of boronic acid compounds have utilized herein to synthesize both cis- and trans-boronic acid mimetics of resveratrol. (FIG. 2)

Resveratrol is classified as a phytoestrogen due to its structural similarity to the synthetic estrogen diethylstilbestrol and its ability to interact with alpha and beta estrogen receptors (ERα and ERβ). (Mol Nutr Food Res, 53: 845, 2009). However in the presence of estrogen (E2), resveratrol can function as an agonist or antagonist with respect to the growth of ER positive (ER+) cells (Proc Natl Acad Sci U.S A, 94, 14138, 1997; Int J Cancer, 104, 587, 2003; J Cell Physiol, 179, 297, 1999). Resveratrol has also been considered a selective ER-modulator (SERM). ((Cancer Res, 61, 7456, 2001; Life Sci, 66, 769, 2000). Therefore, prior to the methods disclosed herein, the anti cancer effect of resveratrol in ER+ breast cancer cells was controversial due to its mixed agonist/antagonist activity.

### BRIEF DESCRIPTION OF THE DRAWING

FIG. 1 shows the chemical structures of ER Agonists and Antagonists.

FIG. 2 shows the design of embodiments of the derivatives disclosed herein.

FIG. 3 shows the growth inhibition curve of compound 1, compound 2, and Resveratrol against MCF-7 breast cancer cells. Results are the mean value of triplicate samples. Cells were seeded in a 96 well plate at a density of 3.5×103 per well for 24 h, then treated with vehicle (DMSO) or with increasing concentrations of indicated compounds (1-100 mM) for 48 hours. Cell viability was finally determined by adding WST-1 reagent and measuring the optical absorbance at 45 nm and 630 nm as described in “Materials and methods”. The IC50 value (the concentration yielding 50% growth inhibition) was obtained from the graph of the log of compound concentration versus the fraction of surviving cells. The IC50 for each cell line was calculated using graph pad prism. Data are expressed as mean (SEM) of triplicate samples. The IC50 value (the concentration yielding 50% growth inhibition) was obtained from the graph of the log of compound concentration versus the fraction of surviving cells. The IC50 for each cell line was calculated using graph pad prism. Data are expressed as mean (SEM) of triplicate sample.

FIG. 4 shows that Compound 2 modulates the expression of cell cycle regulators of the G1 Phase of the cell cycle. MCF-7 cells were treated with the indicated concentrations of compound 2 for 24 hours and 48 hours and cell lysates were analyzed by immunoblotting. Protein expression of positive regulators such as cyclin D1, Cdk4, cyclinE, Cdk2 and phospho-Rb were detected by their specific antibodies. Human β-actin was analyzed as a control of gel loading. Twenty-micrograms of lysate was used for each experimental condition.

FIGS. 5A and 5B show that compound 2 potentiates the apoptotic cell death through the activation of PARP cleavage in MCF-7 cells after 48 h and 72 h incubations of compound 2 and Resveratrol. Western blot analysis of PARP cleavage. A. MCF7 cells were treated with trans-4 for 48 h and 72 h and equal amounts of cell lysate were resolved using SDS-PAGE and analyzed by immunoblot using anti-PARP antibody. The blots were reprobed with anti β-actin antibody to confirm equal protein loading. B. Density of cleaved PARP band (normalized with actin) of trans-4 and resveratrol was determined by densitometry NIH image analysis.

FIGS. 6A and 6B show the analysis of apoptosis induction by Hypodiploid DNA Content (Sub G1) in MCF-7 cell line: Cells in active growth were treated with vehicle and indicated concentration of YK-5-104 and RSV for 24 hours (A) and 48 hours (B), then fixed and the DNA content was determined by labeling with propidium iodide and analyzing by flow cytometry.

FIG. 7 shows the effect of compound 2 on a Multidrug resistance cell line. IC50 value (the concentration yielding 50% growth inhibition) was extrapolated from the graph of the log of compound concentration versus the fraction of surviving cells. The 1050 for each cell line was calculated using graph pad prism. Data are expressed as mean (SEM) of triplicate sample. Results are mean value of triplicate samples.

FIGS. 8A, 8B and 8C show that compound 2 potentiates the flavopiridol mediated inhibition of cell proliferation. MCF-7 cells were treated with 10 μM of trans-4 (A), RSV (B) or 100 nM of flavopiridol (C) for 24 hours followed by treatment with or without flavopiridol (0.005-5 μM) or trans-4 (0.2-100 μM) for 48 hours. A Wst-1 assay was performed to measure the cell viability as mentioned in the methods. GI₅₀ are the average of triplicate samples and the experiment was repeated thrice with identical results.

FIGS. 9A and 9B show the effect of compound 2 on cell cycle distribution in MCF-7 breast cancer cell lines. Actively growing MCF-7 cells were treated with vehicle or indicated concentration of compound 2 and resveratrol for 24 h (Panel A) and 48 h (Panel B), fixed and the DNA content was determined by labeling with propidium iodide followed by analyzing with flow cytometry as described in the experimental section. Results are represented as a mean of triplicate samples. The experiment was repeated twice with identical results.

FIGS. 10A and 10B show the effect of compounds on cell cycle distribution in MCF-7 breast cancer cell lines. A. Synchronized MCF-7 cells were treated with 30 μM of vehicle or cis-4 or trans-4 for different time intervals (16, 24, 48 h), fixed in 70% ethanol and DNA content was determined by labeling with PI followed by flow cytometry analysis. B. Percentage of DNA distribution of trans-4 in G1 phase was presented in a bar graph. Results are represented as a mean of triplicate samples. The experiment was repeated thrice with identical results.

FIG. 11 shows (A) the cell cycle phase and (B) the proteins involved in G1-S Progression.

FIG. 12 shows the effect of compound 2 in ER negative cell lines. IC50 value (the concentration yielding 50% growth inhibition) was extrapolated from the graph of the log of compound concentration versus the fraction of surviving cells. The IC50 for each cell line was calculated using graph pad prism. Data are expressed as a mean (SEM) of triplicate sample. Results are a mean value of triplicate samples.

FIG. 13 shows the irreversibility of trans-4 on MCF-7 breast cancer cell growth inhibition under different conditions. After 24 h post incubation, cells were treated with increasing concentrations of trans-4 (1-100 μM) under three different conditions. In the first method, cells were exposed for 48 hours to trans-4. In the second method cells were exposed to trans-4 for 48 h, washed with serum free media and cultured for an additional 48 h without compound. In the third method, cells were treated every 24 hours to fresh media with trans-4 for 72 h. Cells were harvested at the indicated times and analyzed for cell growth inhibition by the WST-1 method. Values represent the mean±SD of triplicate wells. The experiment was repeated thrice with identical results. Controls were exposed daily to vehicle containing medium (not plotted).

FIG. 14 shows the effect of trans-4 or resveratrol on E2-mediated MCF-7 cell growth. MCF-7 cells were treated with estradiol (E2, 10⁻⁹M) alone or in combination with the indicated concentrations of trans-4 or resveratrol. After 5 days incubation, the DNA content of the treated cells was measured using a DNA fluorescence kit (Bio Rad #170-2480). Results are shown as the mean of triplicate samples±SD. The experiment was repeated twice with identical results.

### DETAILED DESCRIPTION

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Disclosed are materials, compounds, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of R groups are discussed, each and every combination and permutation of the inhibitor and the modifications to its R group that are possible are specifically contemplated unless specifically indicated to the contrary. Thus, if a class of substituents A, B, and C are disclosed as well as a class of substituents D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. DEFINITIONS

In this specification and in the claims which follow, reference will be made to a number of terms which shall be defined to have the following meanings:

1. A, an, the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Activity

As used herein, the term “activity” refers to a biological activity.

3. Binding affinity

The term binding affinity as used herein can be defined as two molecules interacting with a kd of at least 10⁻³, 10⁻⁴, 10⁻⁵, 10⁻⁶, 10⁻⁷, 10⁻⁸, or 10⁻⁹ M or tighter binding.

4. Cell

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other.

5. Clathrate

A compound for use in the invention may form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionised, partially ionised, or non-ionised. For a review of such complexes, see J. Pharm. ScL, 64 (8), 1269-1288, by Haleblian (August 1975).

6. Complex

The term complex as used herein refers to the association of a compound with an other compound, molecule, or composition for which the compound has a binding affinity.

7. Coapplication

“Coapplication” is defined as the application of one or more substances simultaneously, such as in the same formulation or consecutively, within a time frame such that each substance is active during a point when the other substance or substances are active.

8. Compound 1 and YK-5-101

The term “compound 1” and “YK-5-101 (cis)” are interchangeable throughout the specification.

9. Compound 2 and YK-5-104

The term “compound 2” and YK-5-104 (trans)” are interchangeable throughout the specification.

10. Comprise

Throughout the description and claims of this specification, the word “comprise” and variations of the word, such as “comprising” and “comprises,” means “including but not limited to,” and is not intended to exclude, for example, other additives, components, integers or steps.

11. Control

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard.

12. Higher, Increases, Elevates

The terms “higher,” “increases,” “elevates,” or “elevation” or variants of these terms, refer to increases above basal levels, e.g., as compared to a control. The terms “low,” “lower,” “reduces,” or “reduction” or variation of these terms, refer to decreases below basal levels, e.g., as compared to a control. For example, basal levels are normal in vivo levels prior to, or in the absence of, or addition of an agent such as an agonist or antagonist to activity.

13. Inhibit

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

14. Linked

As used herein, the terms “linked”, “operably linked” and “operably bound” and variants thereof mean, for purposes of the specification and claims, refer to fusion, bond, adherence or association of sufficient stability of at least two molecules or moieties to withstand conditions encountered in single molecule applications and/or the methods and systems disclosed herein, such that at least one activity of each individual molecule or the linked composition is preserved or promoted. Examples of linked molecules are a detectable label and nucleotide, between a detectable label and a linker, between a nucleotide and a linker, between a protein and a functionalized nanocrystal; between a linker and a protein; and the like. For example, in a labeled polymerase, the label is operably linked to the polymerase in such a way that the resultant labeled polymerase can readily participate in a polymerization reaction. See, for example, Hermanson, G., 2008, Bioconjugate Techniques, Second Edition. Such operable linkage or binding may comprise any sort of fusion, bond, adherence or association, including, but not limited to, covalent, ionic, hydrogen, hydrophilic, hydrophobic or affinity bonding, affinity bonding, van der Waals forces, mechanical bonding, etc.

15. Linker

The term “linker” and its variants, as used herein, include any compound or moiety that can act as a molecular bridge that operably links two different molecules.

16. Metabolite

The term “metabolite” refers to active derivatives produced upon introduction of a compound into a biological milieu, such as a patient.

17. Optionally

“Optional” or “optionally” means that the subsequently described event or circumstance may or may not occur, and that the description includes instances where said event or circumstance occurs and instances where it does not.

18. Parts by Weight

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

19. Pharmacological Activity

As used herein, the term “pharmacological activity” refers to the inherent physical properties of compound or composition, such as a peptide or polypeptide. These properties include but are not limited to half-life, solubility, and stability and other pharmacokinetic properties.

20. Pharmaceutically Acceptable

By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained.

21. Prevent

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

22. Primers

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

23. Probes

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

24. Pro-Drug

The term “pro-drug or prodrug” is intended to encompass compounds which, under physiologic conditions, are converted into therapeutically active agents. A common method for making a prodrug is to include selected moieties which are hydrolyzed under physiologic conditions to reveal the desired molecule. In other embodiments, the prodrug is converted by an enzymatic activity of the host animal.

25. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

26. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

27. Salt(s) and Pharmaceutically Acceptable Salt(s)

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound disclosed herein with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxyethanebutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (CrC6) halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

The compounds of the invention and their salts may exist in both unsolvated and solvated forms.

28. Solvate

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun, 17:1889-1896 (2004). For a general review of multi-component complexes, see J. K. Haleblian, J. Pharm. Sci. 64(8):1269-88 (1975).

29. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc) mammals, non-human mammals, primates, non-human primates, rodents, birds, reptiles, amphibians, fish, and any other animal. The subject can be a mammal such as a primate or a human. The subject can also be a non-human.

30. Stable

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

31. Tautomer

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

32. Treat, Treating, Treatment

In the context of a subject “Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. Treat in certain contexts herein can also mean to add to or incubate with.

33. Therapeutically Effective

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

34. Weight Percent

A weight percent of a component, unless specifically stated to the contrary, is based on the total weight of the formulation or composition in which the component is included.

35. Chemistry Terms

The term “alkyl” as used herein is a branched or unbranched saturated hydrocarbon moiety. “Unbranched” or “Branched” alkyls comprise a non-cyclic, saturated, straight or branched chain hydrocarbon moiety having from 1 to 24 carbons, 1 to 12, carbons, 1 to 6 carbons, or 1 to 4 carbon atoms. Examples of such alkyl radicals include methyl, ethyl, propyl, butyl, pentyl, hexyl, heptyl, octyl, n-propyl, iso-propyl, butyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”, the later denotes an alkyl radical analogous to the above definition that is further substituted with one, two, or more additional organic or inorganic substituent groups. Suitable substituent groups include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substituted of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

The term “alkenyl” as used herein is an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

The term “alkynyl” as used herein is an alkyl residue as defined above that comprises at least one carbon-carbon triple bond in the backbone of the hydrocarbon chain. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “cycloalkyl” as used herein is a saturated hydrocarbon structure wherein the structure is closed to form at least one ring. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

Moreover, the term “cycloalkyl” as used throughout the specification and claims is intended to include both “unsubstituted cycloalkyls” and “substituted cycloalkyls”, the later denotes an cycloalkyl radical analogous to the above definition that is further substituted with one, two, or more additional organic or inorganic substituent groups that can include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

The term “cycloalkenyl” as used herein is a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like.

The term “alkoxy” as used herein is an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like.

The term “mono-substituted amino” as used herein is a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH3); ethylamino (—NHCH2CH3), hydroxyethylamino (—NH—CH2CH2OH), and the like.

As used herein, the term “azide”, “azido” and their variants refer to any moiety or compound comprising the monovalent group —N3 or the monovalent ion —N3.

The term “di-substituted amino” as used herein is a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

The term “haloalkyl” as used herein an alkyl residue as defined above, substituted with one or more halogens, preferably fluorine, such as a trifluoromethyl, pentafluoroethyl and the like.

The term “haloalkoxy” as used herein a haloalkyl residue as defined above that is directly attached to an oxygen to form trifluoromethoxy, pentafluoroethoxy and the like.

The term “acyl” as used herein is a R—C(O)— residue having an R group containing 1 to 8 carbons. Examples include but are not limited to formyl, acetyl, propionyl, butanoyl, iso-butanoyl, pentanoyl, hexanoyl, heptanoyl, benzoyl and the like, and natural or un-natural amino acids.

The term “acyloxy” as used herein is an acyl radical as defined above directly attached to an oxygen to form an R—C(O)O— residue. Examples include but are not limited to acetyloxy, propionyloxy, butanoyloxy, iso-butanoyloxy, benzoyloxy and the like.

The term “aryl” as used herein is a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “aryl” can themselves be substituted, as described above, if appropriate.

The term “heteroaryl” as used herein is an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

The term “heterocyclyl” or “heterocyclic group” as used herein is a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, oxolane, thiolane, imidazole, oxazole, piperidine, piperizine, morpholine, lactones, lactams, such as azetidiones, and pyrrolidiones, sultams, sultones, and the like. Moreover, the term “heterocyclyl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

The term “halo” or “halogen” refers to a fluoro, chloro, bromo or iodo group.

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

A “moiety” is part of a molecule (or compound, or analog, etc.). A “functional group” is a specific group of atoms in a molecule. A moiety can be a functional group or can include one or functional groups.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

The term “keto group” as used herein is represented by the formula —C(O)R, where R is an alkyl, alkenyl, alkynyl, aryl, aralkyl, cycloalkyl, halogenated alkyl, or heterocycloalkyl group described above.

The term “aldehyde” as used herein is represented by the formula —C(O)H.

The term “carboxylic acid” as used herein is represented by the formula —C(O)OH.

The term “carbonyl group” as used herein is represented by the formula C═O.

The term “ether” as used herein is represented by the formula AOA1, where A and A1 can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

The term “sulfo-oxo group” as used herein is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

### B. RESVERATROL AND ITS ANALOGS

Resveratrol and its analogs, pterostilbene (3,5-dimethoxy-4′-hydroxy-trans-stilbene), TMS (3,4′,5-reimwrhoxzy-trans-stilbene), 3,4′,4-DH-5-MS (3,4′-dihydroxyδ-methoxy-trans-stilbnene) and 3,5-DH-4′ MS (3,5-dihydroxy-4′-ethoxy-trans-stilbene), have been shown to have chemopreventative activity against cardiovascular disease and a variety of cancers in model systems. See, Jong et al., Science, 275 (5297), 218-220 (1997), Rimando et al., J. Agric. Food Chem., 50 (12), 3453-3457 (2002), Aggarwal et al., Biochem. Pharmacol., 71 (10), 1397-1421 (2006) and Baur et al., Nat. Rev. Drug Discov., 5 (6), 493-506 (2006). Stilbenes have been found in some berries (e.g., blueberries, cranberries, sparkleberries, lingonberries, grapes). See, Rimando et al., J. Agric. Food Chem., 50 (15), 4713-4719 (2004). Resveratrol, a natural product found in the skin of red grapes has received interest for potential anticancer activity. Thus consumption of these small fruits may help improve health. Dietary black raspberries significantly suppressed the N-nitrosomethylbenzylamine (NMBA)-induced rat esophageal carcinogenesis. Chen et al., Cancer Res., 66 (5), 2853 2859 (2006). The discovery of resveratrol as a cancer preventive agent has fostered interest in testing for the cancer preventive activity of other naturally occurring stilbenes in many laboratories.

The chemical structure of resveratrol is very similar to that of the synthetic estrogen agonist, diethylstilbestrol, suggesting that resveratrol might also function as an estrogen agonist. However, in cell culture experiments resveratrol acts as an estrogen agonist under some condition, and an estrogen antagonist under other conditions. In estrogen receptor-positive breast cancer cells, resveratrol acted as an estrogen agonist in the absence of the endogenous estrogen 17-beta-estradiol, but acted as an estrogen antagonist in the presence of 17-beta estradiol. At present it appears that resveratrol has the potential to act as an estrogen agonist or antagonist, depending upon factors such as cell type, estrogen receptor isoform OR alpha or ER beta), and the presence of endogenous estrogens.

### C. COMPOSITIONS AND METHODS

Disclosed herein are boronic acid analogs of resveratrol which were designed and synthesized as new clinical compounds related to cancer, such as breast cancer. Trans-boronic acid resveratrol showed more potent cytotoxic effects against estrogen dependent MCF-7 cells than resveratrol. Cell cycle and western blot analysis demonstrated that the trans analogs inhibits the G1 cell cycle. This can provide a rationale for the increased potency of the trans-boronic acid analogs in MCF-7 cells as compare to resveratrol.

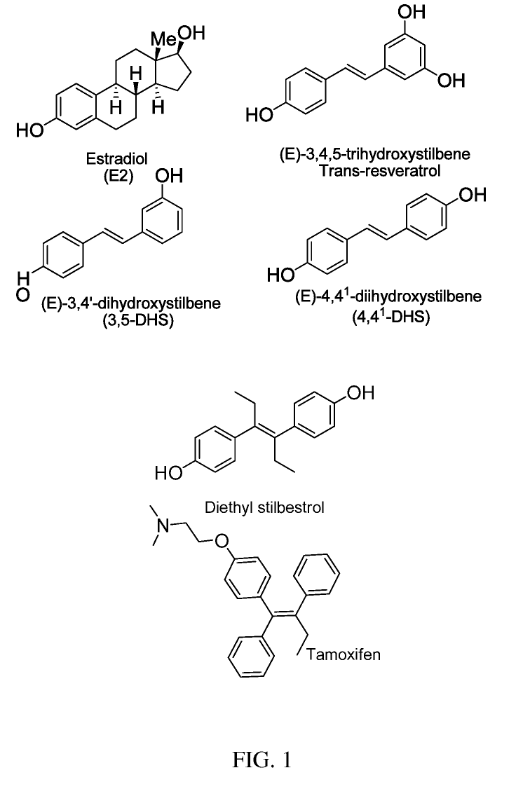

One approach to inhibiting estrogen-responsive genes is to block the receptors with antagonist from natural or semi-synthetic origin. (FIG. 1)

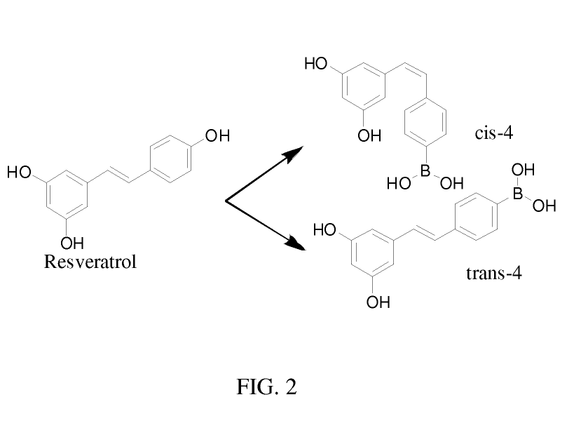

Disclosed are compositions which have better activity than Resveratrol for treating breast cancer. In certain embodiments the trans configuration of the disclosed compositions (for example, Compound 2, Shown in FIG. 2) has 3-fold more activity than Resveratrol. The cis configuration has less activity than Resveratrol. Generally, cis configurations of Stilbenes are active (See for example, Nakamura et al., Chem. Med. Chem. 1:729-740 (2006).

Generally, compound 2 is more stable than Resveratrol (R), the solubility of compound 2 is higher than R due to the B(OH)₂ gp, and the mechanism of action of Compound 2 is different from R. R works at the G2/M1 phase of cell cycle whereas Compound 2 arrests the cycle at the G1 phase of the cell cycle. Unlike R, Compound 2 demonstrates an irreversible effect. Irreversibility is desirable from a clinical perspective because this lowers the needed therapeutic dose as compared to non-irreversible compounds, allowing for example, a therapeutic dose that can be given only once. Unlike R, Compound 2 can work on multidrug resistant cell lines as well as on estrogen-dependent cell lines.

Derivatives of Resveratrol disclosed herein are for example,

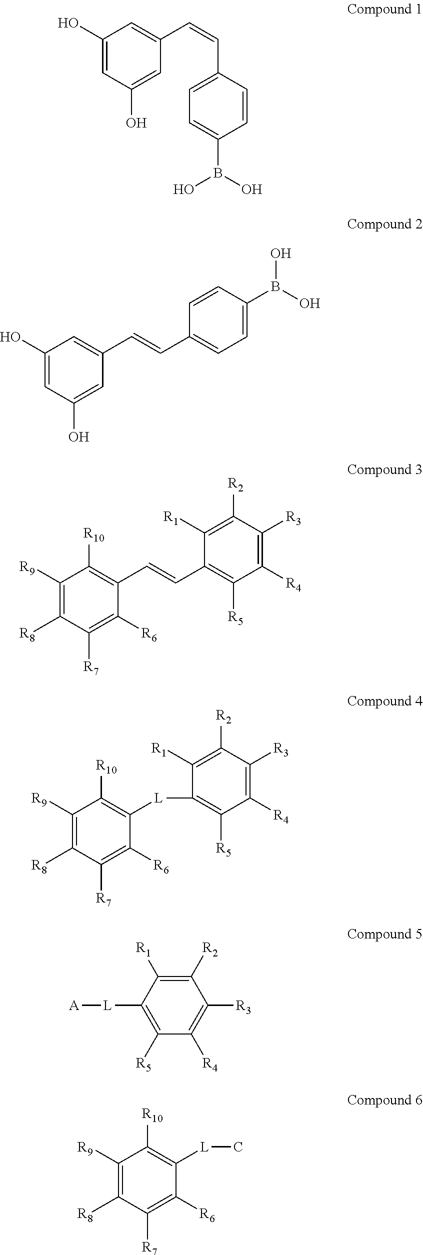

Disclosed herein are compounds of structure A-L-C, or a pharmaceutically acceptable salt, prodrug, clathrate, tautomer or solvate thereof, wherein:
A is substituted or unsubstituted cylcoalkyl, aryl, heteroaryl, heterocyclyl;
L is present or absent, if present L is a linker; and
C is substituted or unsubstituted cylcoalkyl, aryl, heteroaryl, heterocyclyl, wherein at least one position in the compound is substituted with —B(OH)₂, and at least one position is substituted with alkoxy, alkoxydialkylamino or hydroxyl.

Also disclosed herein are compositions or a pharmaceutically acceptable salt, prodrug, clathrate, tautomer or solvate thereof comprising, compounds of structure A-L-C, or a pharmaceutically acceptable salt, prodrug, clathrate, tautomer or solvate thereof, wherein:

A is substituted or unsubstituted cylcoalkyl, aryl, heteroaryl, heterocyclyl;

L is present or absent, if present L is a linker; and

C is substituted or unsubstituted cylcoalkyl, aryl, heteroaryl, heterocyclyl, wherein at least one position is substituted with —B(OH)₂, and at least one position is substituted with alkoxy, alkoxydialkylamino or hydroxyl.

Also disclosed herein are methods of treating cancer comprising, administering to a subject in need of treatment a composition comprising, a compound of structure A-L-C, or a pharmaceutically acceptable salt, prodrug, clathrate, tautomer or solvate thereof, wherein:
A is substituted or unsubstituted cylcoalkyl, aryl, heteroaryl, heterocyclyl;
L is present or absent, if present L is a linker; and
C is substituted or unsubstituted cylcoalkyl, aryl, heteroaryl, heterocyclyl, wherein at least one position is substituted with —B(OH)₂, and at least one position is substituted with alkoxy, alkoxydialkylamino or hydroxyl.

In some forms, A can be cylcoalkyl, aryl, heteroaryl, heterocyclyl, L can be a linker or nothing, and C can be cylcoalkyl, aryl, heteroaryl, heterocyclyl, wherein both meta positions of A relative to L can be —B(OH)₂, carboxylic acid, a mild lewis acid, a strong acid, or a weak acid, hydroxyl, or C1-C4 alkoxy and wherein the para position of C relative to L can be —B(OH)₂, carboxylic acid, a mild lewis acid, a strong acid, or a weak acid, hydroxyl, or C1-C4 alkoxy, and wherein zero or more remaining reactive positions on A and C can be a halogen.

In some forms A can be substituted phenyl.

In some forms L can be a linker. In some forms L is present or absent.

In some forms, C can be substituted phenyl.

In some forms, structure A-L-C can have the structure

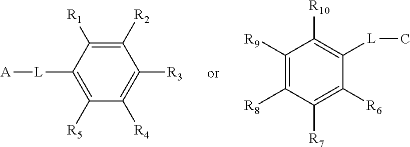

wherein:

R¹, R², R³, R⁴, R⁵, R⁶, R⁷, R⁸, R⁹ and R¹⁰ can independently be hydrogen, —B(OH)₂, mild lewis acid, strong acid, weak acid, alkyl, alkenyl, alkynyl, halo, alkoxy, amino, alkylamino, dialkylamino, cyano, nitro, formyl, carboxyl, alkoxycarbonyl, aminocarbonyl, alkylaminocarbonyl, dialkylamino carbonyl, haloalkyl, haloalkloxy, haloalkylamino, di(haloalkyl)amino or sugars;

R⁸ and R⁹ can optionally be cyclized to form cylcoalkyl, aryl, heteroaryl or heterocyclyl, which can optionally be substituted with —B(OH)₂, mild lewis acid, strong acid, weak acid, alkyl, alkenyl, alkynyl, halo, alkoxy, amino, alkylamino, dialkylamino, cyano, nitro, formyl, carboxyl, alkoxycarbonyl, aminocarbonyl, alkylaminocarbonyl, dialkylamino carbonyl, haloalkyl, haloalkloxy, haloalkylamino, di(haloalkyl)amino or sugars;

L can be present or absent, if present L can be C₁-C₆ alkyl, C₂-C₆ alkenyl, aryl, heteroaryl, cycloalkyl, heterocyclyl, —P-Q-S—, wherein P can be C₁-C₆ alkyl, C₂-C₆ alkenyl, aryl, heteroaryl, cycloalkyl or heterocyclyl, Q can be —N(R¹¹)—, —O—, —S—, —C(O)—, wherein R¹¹ can be hydrogen or C₁-C₃ alkyl, S can be present or absent, if present S can be C₁-C₆ alkyl, C₂-C₆ alkenyl, aryl, heteroaryl, cycloalkyl or heterocyclyl.

In some forms, R¹, R², R⁴, R⁵, R⁶, R⁸, and R¹⁰ can independently be H, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl.

In some forms, the structure can be

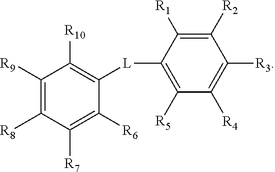

In some forms, R³ can be —B(OH)₂, hydroxyl or C₁-C₃ alkoxy. In some forms, R³ can be —B(OH)₂ or C₁ alkoxy. In some forms, R³ can be —B(OH)₂.

In some forms, R¹, R⁵, R⁶, R¹⁰ can be hydrogen.

In some forms, R² and R⁴ can independently be hydrogen or C₁-C₃ alkoxy. In some forms R² and R⁴ can be hydrogen. In some forms, R² and R⁴ can be C₁ alkoxy.

In some forms, R⁷ and R⁹ can independently be hydrogen, —B(OH)₂, hydroxyl, C₁-C₃ alkoxy or C₁-C₃ alkoxydialkylamino. In some forms, R⁷ and R⁹ can independently be hydroxyl, C₁-C₃ alkoxy or C₁-C₃ alkoxydialkylamino. In some forms, R⁷ and R⁹ can be identical moieties. In some forms, R⁷ and R⁹ can be different moieties. In some forms, R⁷ and R⁹ can be Cᵢ alkoxy. R⁷ and R⁹ can be hydroxyl.

In some forms, R³ can be —B(OH)₂, hydroxyl or C₁-C₃ alkoxy; R² and R⁴ can independently be hydrogen or C₁-C₃ alkoxy; R⁷ and R⁹ can independently be hydrogen, —B(OH)₂, hydroxyl, C₁-C₃ alkoxy or C₁-C₃ alkoxydialkylamino; and R⁸ can be hydrogen or C₁-C₃ alkoxy.

In some forms, L can be absent.

In some forms, L can be present. In some forms L can be C₁-C₆ alkyl, C₂-C₆ alkenyl, aryl, heteroaryl, cycloalkyl, heterocyclyl, —P-Q-S—, wherein P can be C₁-C₆ alkyl, C₂-C₆ alkenyl, aryl, heteroaryl, cycloalkyl or heterocyclyl, Q can be —N(R¹¹)—, —O—, —S—, —C(O)—, wherein R¹¹ can be hydrogen or C₁-C₃ alkyl, S can be present or absent, if present S can be C₁-C₆ alkyl, C₂-C₆ alkenyl, aryl, heteroaryl, cycloalkyl or heterocyclyl. In some forms, L can be C₂-C₆ alkenyl. In some forms L can be comprise —C(O)—. In some forms L can comprise C₃-C₆ cycloalkyl. In some forms L can comprise C₂-C₆ alkenyl and —C(O)—. In some forms, L can comprise C₂-C₅ heterocyclyl. In some forms, L can comprise C₁-C₆ alkyl and aryl, heteroaryl, cycloalkyl or heterocyclyl. In some forms, L can comprise C₂ alkenyl. In some forms, L can be:

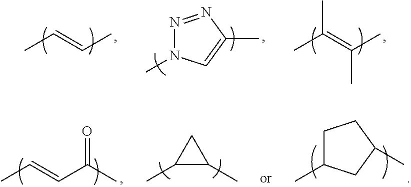

In some forms, the structure A-L-C can be:

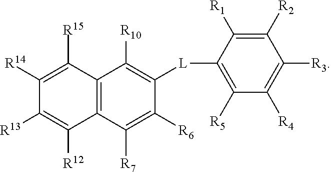

In some forms, R¹², R¹³, R¹⁴ and R¹⁵ can independently be hydrogen, —B(OH)₂, mild lewis acid, strong acid, weak acid, alkyl, alkenyl, alkynyl, halo, alkoxy, amino, alkylamino, dialkylamino, cyano, nitro, formyl, carboxyl, alkoxycarbonyl, aminocarbonyl, alkylaminocarbonyl, dialkylamino carbonyl, haloalkyl, haloalkloxy, haloalkylamino, di(haloalkyl)amino or sugars. In some forms R¹³ and R¹⁵ can be hydrogen. In some forms R¹² and R¹⁴ can independently be —B(OH)₂, hydroxyl, C₁-C₃ alkoxy or C₁-C₃. alkoxydialkylamino. In some forms, R¹³ and R¹⁵ can be hydrogen and R¹² and R¹⁴ can independently be —B(OH)₂, hydroxyl, C₁-C₃ alkoxy or C₁-C₃.

In some forms, structure A-L-C can have the structure:

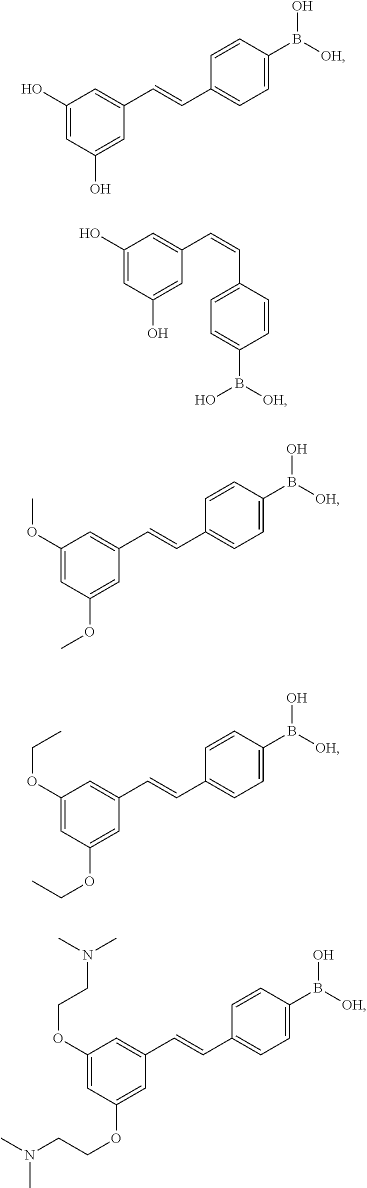

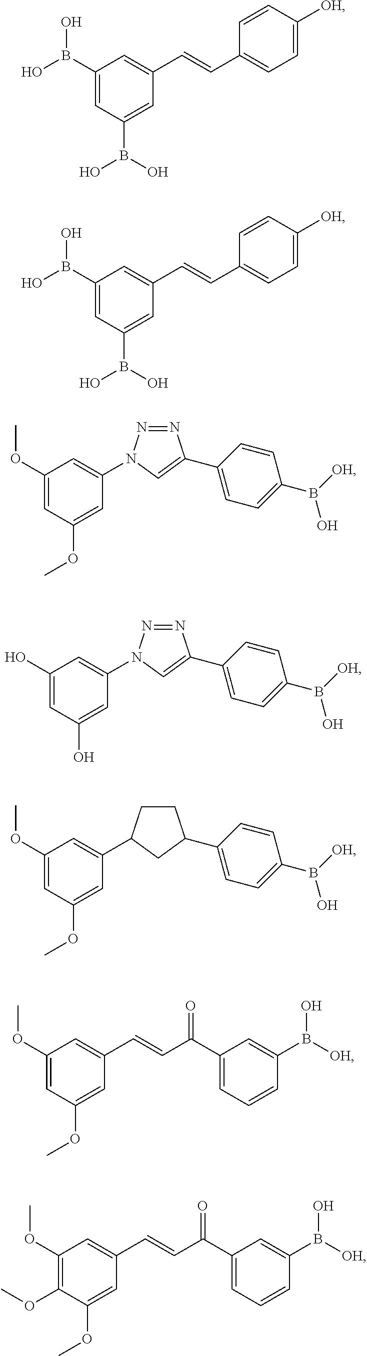

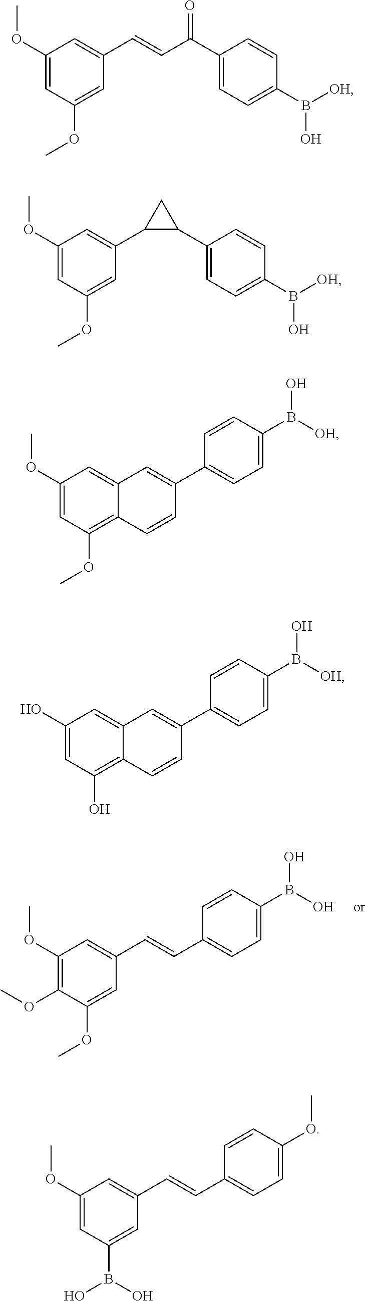

In some forms the compound can be trans. In some forms the compound can be cis. In some forms the compound is isolated trans. In some forms the compound can be isolated cis.

In some forms, the subject could have been assayed for cancer or a risk of cancer. In some forms, the subject can be at risk of having cancer. In some forms, the subject could have been diagnosed with cancer. In some forms, the cancer can be any cancer expressing ER. In some forms, the cancer can be breast cancer. In some forms, the subject can be assayed for the presence of cancer following administration of the composition.

In some forms, the composition can be administered in a therapeutically effective amount. In some forms, the composition can comprise a pharmaceutically acceptable carrier.

Disclosed are compounds having the structure A-L-C. In certain embodiments, A is cylcoalkyl, aryl, heteroaryl, heterocyclyl, L is a linker or nothing, and C is cylcoalkyl, aryl, heteroaryl, heterocyclyl.

In compounds 3, and 4, R3 is where the boronic acid would go. R9 and R7 is where the hydroxyl groups would go.

In certain embodiments, for Compound 3:

R1, R2, R4, R5, R6, R8, and R10 can independently be H, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl.

While R3, can be a functional group as described for R1 and R3 is preferably boronic acid (B(OH)₂) carboxylic acid, a mild lewis acid, a strong acid, or a weak acid. In certain embodiments, R3 is boronic acid.

While R7 and R9 can independently be a functional group as described for R1, R7 and R9 in certain embodiments can be boronic acid (B(OH.2)2), carboxylic acid, a mild lewis acid, a strong acid, or a weak acid. In certain embodiments, R7 and R9 are a hydroxyl.

In certain embodiments, the linker is not necessarily present. Boronic acid is a weak/mild Lewis acid, and can be changed for other like acids (carboxylic acid etc). In certain embodiments, boronic acid and hydroxyl groups can be R2 and R4. Click chemistry can be used for linking A and L.

Disclosed are compounds comprising, a structure A-L-C, wherein A is cylcoalkyl, aryl, heteroaryl, heterocyclyl, L is a linker or nothing, and C is cylcoalkyl, aryl, heteroaryl, heterocyclyl, wherein both meta positions of A relative to L are boronic acid (B(OH.2)2), carboxylic acid, a mild lewis acid, a strong acid, or a weak acid, hydroxyl, or C1-C4 alkoxy and wherein the para position of C relative to L is preferably boronic acid (B(OH.2)2), carboxylic acid, a mild lewis acid, a strong acid, or a weak acid, hydroxyl, or C1-C4 alkoxy, and wherein zero or more remaining reactive positions on A and C can be a halogen.

Also disclosed are compounds, wherein the L is a C2-C6 alkenyl.

Also disclosed are compounds, wherein the structure comprises the structure

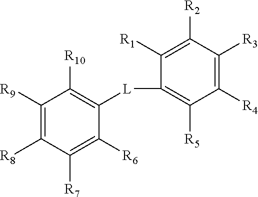

Disclosed are compounds, wherein R1, R2, R4, R5, R6, R8, and R10 can independently be H, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl; wherein R3 is boronic acid; wherein R7 and R9 are hydroxyl; wherein the L is a C2 alkenyl; wherein R1, R2, R4, R5, R6, R8 and R10 are hydrogen, and/or any combination or alone of these or any other characteristic disclosed herein.

Disclosed are compositions comprising any of the compounds disclosed herein.

Also disclosed are complexes comprising any of the compositions or compounds disclosed herein and a cell, wherein the cell expresses ER, wherein the cell is a cancer cell, wherein the cell is a breast cancer cell and/or any combination or alone of these or any other characteristic disclosed herein.

Disclosed are complexes comprising any of the compositions or compounds disclosed herein and ER or homolog, and/or any combination or alone of these or any other characteristic disclosed herein.

Also disclosed are methods comprising administering any of the compositions or compounds to a subject.

Disclosed are methods, wherein the subject has been assayed for cancer or a risk of cancer, wherein the subject has been treated for cancer, wherein the cancer expresses ER, wherein the cancer is breast cancer, wherein the subject is in need of treatment for cancer, wherein the subject is assayed for the presence of cancer following administration of the composition, and/or any combination or alone of these or any other characteristic disclosed herein.

1. General Compositions

i. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue. (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue. (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed. (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

a. Therapeutic Uses

Effective dosages and schedules for administering the compositions may be determined empirically, and making such determinations is within the skill in the art. The dosage ranges for the administration of the compositions are those large enough to produce the desired effect in which the symptoms disorder is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing a cancer, such as prostate cancer, the efficacy of the therapeutic antibody can be assessed in various ways well known to the skilled practitioner

The compositions that inhibit disclosed ER and cancer, such as breast cancer, interactions disclosed herein may be administered as a therapy or prophylactically to patients or subjects who are at risk for the cancer or breast cancer.

ii. Compositions Identified by Screening with Disclosed Compositions/Combinatorial Chemistry

a. Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, disclosed ER and Compounds 1-6s, are also disclosed. Thus, the products produced using the combinatorial or screening approaches that involve the disclosed compositions, such as, disclosed ERs and Compounds 1-6, are also considered herein disclosed.

It is understood that the disclosed methods for identifying molecules that inhibit the interactions between, for example, disclosed ERs and Compounds 1-6 can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well. Combinatorial chemistry includes but is not limited to all methods for isolating small molecules or macromolecules that are capable of binding either a small molecule or another macromolecule, typically in an iterative process.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514). Libraries using the disclosed compounds, such as Compounds 1-6 can be made.

As used herein combinatorial methods and libraries included traditional screening methods and libraries as well as methods and libraries used in interactive processes.

b. Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, disclosed ERs and Compounds 1-6, are also disclosed. Thus, the products produced using the molecular modeling approaches that involve the disclosed compositions, such as, disclosed ERs and Compounds 1-6s, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### D. EXAMPLES

### 1. Summary

Disclosed herein are compounds and uses of the boronic acid biomimetics of resveratrol in cancer therapy. More particularly, the compounds and methods relate to synthesis of trans boronic acid biomimetics of resveratrol (trans-4) and their biological evaluation against the estrogen dependant breast cancer MCF-7 cell line. The disclosed boronic acid biomimetics of resveratrol demonstrated improved cellular toxicity against estrogen dependant human breast cancer cells compared to that of resveratrol. Trans-4 specifically induces G1 cell cycle arrest, which coincides with marked inhibition of cell cycle proteins and a greater pro-apoptotic effect. Furthermore, these compounds exhibit the irreversible anti-proliferative effect with undiminished activity against a multidrug resistance cell line, which indicates it as a viable therapeutic agent for treating cancer, such as breast cancer. Combination treatments with flavopiridol, a known preclinical cdk inhibitor studied in breast cancer, show greatly enhanced potency for inhibiting cancer cell proliferation Altogether the results demonstrate that trans-4 inhibits breast cancer cells by a different mechanism of action than resveratrol (s-phase arrest), and provide information to aid in the design of new anticancer agents that incorporate boronic acid into the basic chemical structure.

Also disclosed are methods for preparing different compounds that may have useful significance in cancer therapy.

### 2. Chemistry

The design strategy for the disclosed compositions is shown in FIG. 2. YK-5-101 is referred to herein as Compound 1. YK-5-104 is referred to herein as compound 2. A set of compounds which are boronic acid derivatives of Resveratrol are also disclosed herein.

The strategy used to synthesize the boronic acid resveratrol analogs cis-4 and trans-4 is outlined in Schemel. Briefly, 5-(bromomethyl)-1, 3

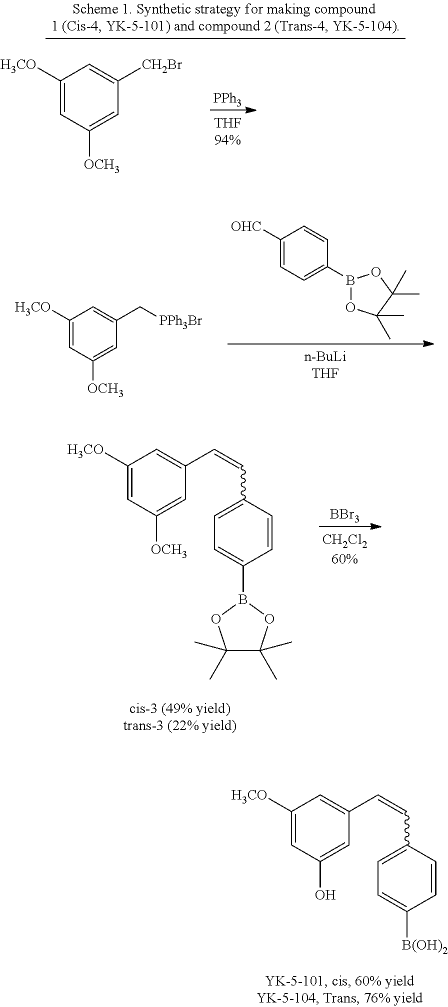

dimethoxybenzene was treated with triphenylphosphine to yield the phosphonium salt 2 with 94% yield. This was followed by a Wittig coupling of 2 with 4-formylphenyl boronic acid pinacol ester in the presence of n-BuLi and resulted in a mixture of stilbenes 3 (in a 2:1 cis/trans ratio). The cis/trans mixture was purified and isomers separated by flash column chromatography. Conversion of the separated cis and trans isomers to final products cis-1 and trans-1 boronic acids was accomplished by treating cis-3 and trans-3 independently with borontribromide to afford fully deprotected products. The purity of all the final target compounds were confirmed by reverse phase HPLC analysis using two different mobile phases (A: 10%-40% acetonitrile in H₂O(v/v), flow rate at 1 mL/min over 20 mins; method B, 8%-40% methanol in H₂O(v/v), flow rate at 1 mL/min over 20 mins) In both methods compound purity were found to be 93.27% and 92.36% respectively. Throughout this study the trans boronic acid derivative of resveratrol is mentioned as compound 2

The design strategy for the synthesis of trans 8, -12 and -13 compounds are shown in Scheme 2.

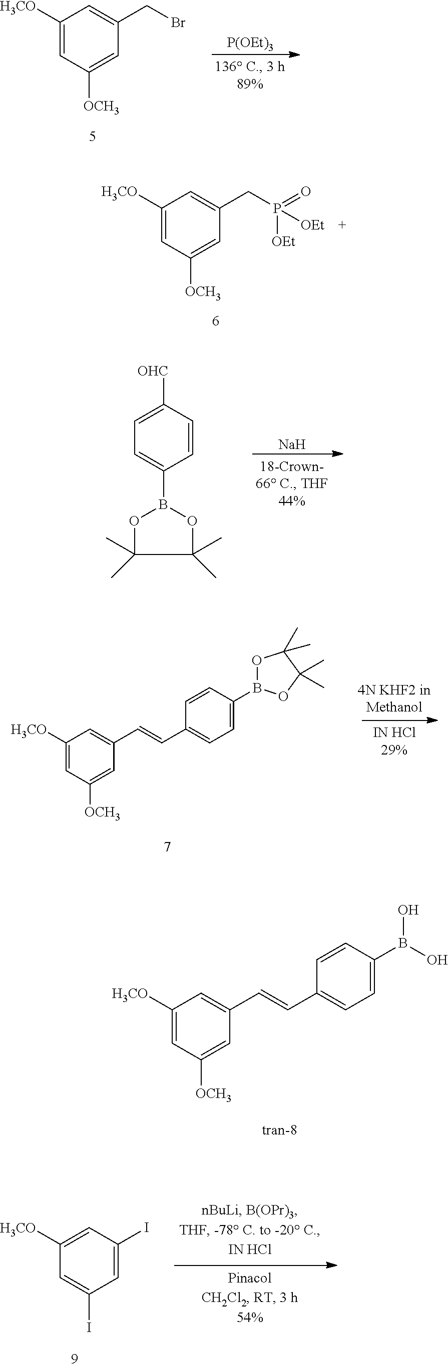

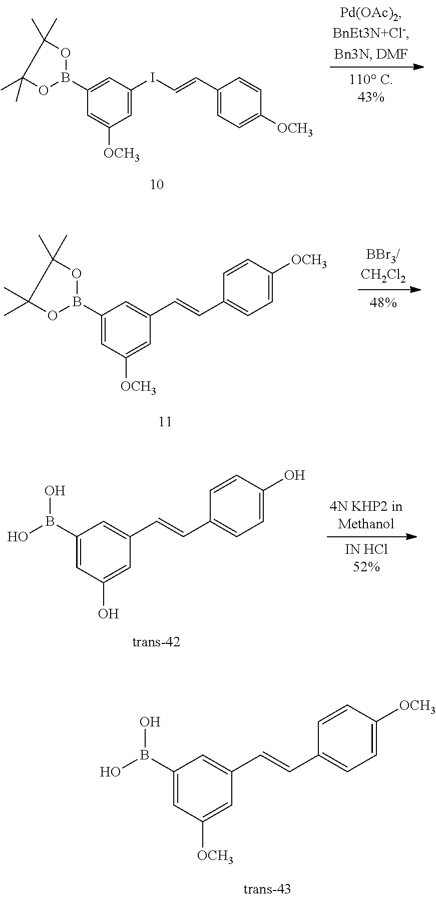

### 3. Materials and Methods

General Methods NMR spectra were recorded using a Varian-400 spectrometer for ¹H (400 MHz) and ¹³C (100 MHz). Chemical shifts (δ) are given in ppm downfield from tetramethylsilane, as internal standard, and coupling constants (J-values) are in hertz (Hz). Purifications by flash chromatography were performed. Analytical high pressure liquid chromatography (HPLC) and liquid chromatography/mass spectrometry (LC/MS) analyses were conducted using Shimadzu LC-20AD pumps and a SPD-20A UV-vis detector. Reverse phase HPLC was performed on Restek's Ultra IBD C18 (5 μm, 4.6×50 mm) using two Shimazu LC-20AD pumps and a SPD-20A-vis detector set at 330 nm: Method A, 10%-40% acetonitrile in H₂O(v/v), flow rate at 1 mL/min over 20 mins; method B, 8%-40% methanol in H₂O(v/v), flow rate at 1 mL/min over 20 mins High-resolution mass spectra (HMRS) were recorded on a QSTAR Elite mass spectrometer.

i. Cell Lines and Culture:

ER-Positive (MCF-7) human breast carcinoma cell line was used in this study. The human breast cell lines MCF-7 (HTB-22) were provided by the tissue culture core facility at Lombardi Comprehensive Cancer Center, Georgetown University Medical Center. MCF-7^{MDR} (CL 10.3) cells were a gift from Dr. Robert Clarke from the Georgetown University Medical Center. MCF-7, and CL 10.3 cells were maintained in DMEM (Biofluids, Frederick, Md.) supplemented 10% heat inactivated fetal bovine serum (FBS), 2 mM L-glutamine, and 50 μg/mL each of antibiotics, namely penicillin, streptomycin, and neomycin at 37° C. in a humidified incubator containing 5% CO₂.

ii. Cell Growth Assay (WST-1 Assay)

The effect of cis-4 and trans-4 or resveratrol on cell growth was determined by WST-1 assay. Briefly, cells were seeded into a 96-well plate at 3,500 cells per well in DMEM containing 10% FBS. After overnight incubation, cells were treated with the compounds (1-100 μM) for 48 h and 72 h at 37° C. Control cells were treated with an equal amount of DMSO. After the indicated incubation time, cell viability was measured by WST-1 assay according to the manufacturer's instructions (Roche). Briefly, 20 μL of WST-1 solution was added in each well and incubated for 2-4 hours. The water soluble tetrazolium salt of WST-1 is converted into orange formazan by dehydrogenase in the mitochondria of living cells. The formazan absorbance, which correlates to the number of living cells, was recorded at wavelengths of 450 nm and 630 nm using a microplate reader (Ultramark, Microplate Imaging System, Bio-Rad). The GI₅₀ was calculated from the graph of the log of compound concentration versus the fraction of the surviving cells.

iii. Cell Cycle Analysis

The effect of cis-4, trans-4 or resveratrol on cell cycle progression was analyzed by flow cytometry. Cells were seeded in 6-well plates and treated with 30 μM of compound 1 or 2 at different time intervals (16, 24, 48 h). Cells were trypsinized, centrifuged at 2000 rpm and cell pellets were collected. Pellets were washed with 1×PBS, permeabilized with 70% (v/v) ethanol, resuspended in 1 ml of PBS containing 1 mg/ml RNase and 50 mg/ml propidium iodide, incubated in the dark for 30 min at room temperature, and analysed by a FACSort Flow Cytometer (Becton Dickinson, San jose, CA). The cell cycle distribution was evaluated on DNA plots using the Modfit software (Verity softwarehouse, Topsham, Me.).

iv. Western Blot Analysis

Western blotting was performed as previously published (J Biol Chem 2004, 279, 38903-38911). In brief, cell pellets were collected at the indicated times after treatment with compounds, suspended in 100 μl, of lysis buffer (50 mM Tris-HCl, 150 mM NaCl, 1 mM EGTA, 1 mM EDTA, 20 mM NaF, 100 mM Na3VO4, 0.5% NP-40, 1% Triton X-100, 1 mM PMSF, 5 μg/ml aprotinin, 5 μg/ml leupeptin), vortexed twice and incubated in an ice bath for 30 min Lysates were cleared by centrifugation at 12000 rpm for 15 min at 4° C. and protein was estimated by detergent compatible BCA protein assay kit (Pierce). Equivalent amounts of total proteins were resolved by SDS-PAGE (10%) and transferred to PVDF membranes. Membranes were blocked by 5% non-fat powdered milk in TBST overnight. Membranes were incubated with the indicated primary antibodies (Rabbit polyclonal antibodies Cdk2 (SC-163) and Cdk4 (SC-260), mouse monoclonal Cyclin E was obtained from santa cruz, mouse monoclonal Cyclin D was obtained from santa cruz, mouse monoclonal pRb was obtained from BD-Pharmengin, Anti-β-actin was obtained from sigma) for 2 hours followed by HRP-conjugated secondary antibodies for 1 hr and developed using enhanced chemiluminescence (Perkin Elmer). For pRb protein, 6% acrylamide SDS-PAGE was used.

v. Cell Growth Assay (Reversible/Irreversible) for Trans-4

To determine whether the effect of trans-4 was reversible or irreversible, MCF-7 cells were treated with trans-4 under three different methods or conditions. In Method 1, cells were treated with trans-4 continuously for 48 h. In Method 2, cells were treated with trans-4 for 48 h and incubated further in fresh media without trans-4 for an additional 48 h. In Method 3, cells were treated with trans-4 for 72 h with a change in media containing fresh trans-4 after every 24 h. Cell viability was measured at the indicated times by WST-1 assay according to the above mentioned protocol.

vi. Measuring the DNA Content in Estrogen Mediated MCF-7 Cell Growth

To examine the effect of resveratrol or trans-4 on E2-mediated MCF-7 cell growth, MCF-7 cells were cultured in estrogen-depleted media (phenol-red free modified Eagle's medium supplemented with 10% charcoal stripped FBS) for 4 days, changing the media every 24 hours. Cells were then seeded in 24 well plates at 10000 cells per well in 1 ml of estrogen depleted media. After 24 hours incubation, media were changed and contained the indicated concentration of resveratrol, trans-4, 17β-estradiol (E2) alone, or combinations of resveratrol with 17β-estradiol or trans-4 with 17β-estradiol. Cells were incubated at 37° C. for an additional 4 days. Media were removed and cells were frozen at −78° C. overnight and the DNA content in each well was measured according to the manufacturer's protocol (DNA Quantification Kit, Bi0-Rad). Results are represented as mean of triplicate sample and repeated the experiment with identical results.

vii. Test Compounds:

Resveratrol was purchased from Sigma (St. Louis, Mo.) and cis and trans boronic acid derivatives of resveratrol (compounds 1 and 2) were prepared as described below. Compounds were dissolved in DMSO at 50 mM concentration, stored at −20° C., and diluted in serum free medium immediately before use. All experiments were performed in 5% media

### a. 3,5-Dimethoxybenzyltriphenylphosphonium bromide

Triphenylphosphine (6.5 g, 24.7 mmol) was added to a solution of 5-(bromomethyl)-1,3-dimethoxybenzene (4.4 g, 19.0 mmol) in dry THF (30 ml). The mixture was refluxed with stirring for 24 h. The resulting white solid was filtered and washed with ether/hexane to afford a white solid 2 (8.8 g, 94%): ¹H NMR (CDCl₃, 400 MHz) δ 7.70 (m, 9H), 7.59 (m, 6H), 6.29 (m, 2H), 6.23 (m, 2H), 5.22 (d, 2H, J=14.4), 3.46 (s, 6H).

### b. Z-2[4-(3,5-dimethyoxystyryl)phenyl]-4,4,5,5-tetramethyl-1,3,2-dioxaborolane (Cis-3)

Phosphonium bromide 2 (3.0 g, 6.08 mmol) was suspended in dry THF and cooled to −78° C. n-BuLi (3.8 ml, 6.08 mmol, 1.6 M in hexane) was added slowly with stiffing. The mixture was stirred at −78° C. for 3 h, and 4-formylphenyl boronic acid pinacol ester (1.41 g, 6.08 mmol) in 5 ml THF was added dropwise. The reaction temperature was maintained at −78° C. for another hour, and the mixture was warmed to room temperature. The reaction mixture was stirred overnight. The mixture was poured into saturated NH₄Cl (20 ml) and extracted with EtOAc. The extracts were combined, washed with brine and dried over Na₂SO₄. The organic layer was filtered and rotary concentrated to give the crude mixture of cis/trans stilbene 3. Flash column chromatography using 5% EtOAc/hexane eluted the cis-stilbene 3 (R_{f}=0.52, Hex/EtOAc=5/1) as a semi solid (1.1 g, 49%). ¹H NMR (CDCl₃, 400 MHz) δ 7.746 (d, 2H, J=8.0 Hz), 7.32 (d, 2H, J=8.4 Hz), 6.619 (d, 1H, J=12.4 Hz), 6.56 (d, 1H, J=12.4 Hz), 6.44 (m, 2H), 6.34 (m, 2H), 3.63 (s, 6H), 1.35 (s, 12H), 3.71 (s, 6H); ¹³C NMR (100 MHz) δ 158.62, 138.65, 137.46, 133.31, 129.69, 129.31, 127.10, 106.03, 83.53, 55.57, 26.09.

### c. E-2[4-(3,5-dimethyoxystyryl)phenyl]-4,4,5,5-tetramethyl-1,3,2-dioxaborolane (Trans-3)

Flash column as described above yielded stilbene trans-3 (R_{f}=0.49, Hex/EtOAc=5/1) as a pale yellow semi solid (0.5 g, 22%). ¹H NMR (CDCl₃, 400 MHz) δ 7.82 (d, 2H, J=8.0), 7.51 (d, 2H, J=8.0 Hz), 7.10 (dd, 2H, J=16.8, 16.8 Hz), 6.68 (m, 2H). 6.72 (m, 1H), 3.82 (s, 6H), 1.36 (s, 12H); ¹³C NMR (100 MHz) δ 160.17, 139.50, 138.89, 134.96, 129.03, 125.88, 105.15, 100.87, 84.77, 56.99, 77.92, 56.99, 56.98, 27.25.

### d. [(Z)-2-(3,5-dimethoxyphenyl)vinyl]phenylboronic Acid (Cis-4)

Cis-3 (0.4 g, 1.09 mmol) was dissolved in dry CH₂Cl₂ (5 ml) and cooled to −78° C., and BBr₃ (10 ml, 10 mmol, 1.0 M in CHCl₂) was added dropwise. The resulting mixture was stirred at −78° C. for another 1.5 h. The mixture was warmed to room temperature and stirred overnight. The reaction was quenched with H₂O (10 ml). Concentrated and applied to flash column chromatography using 10% CHCl₂/MeOH eluted the cis-1 (R_{f}=0.5, CHCl₂/MeOH=9/1) as a soft solid (0.16 g, 60%). ¹H NMR (CDCl₃, 400 MHz) δ 9.11 (s, 2H), 7.97 (s, 2H), 7.62 (d, 2H, J=8.4), 7.17 (d, 2H, J=8.0 Hz), 6.45 (dd, 2H, J=12.4, 12.8 Hz), 6.05 (m, 3H); ¹³C NMR (100 MHz) δ 159.30, 140.73, 134.71, 134.45, 131.99, 131.18, 131.06, 129.28, 108.43, 102.70. HPLC: Method A, retention time=13.28 min; Method B, retention time=11.32 min; HRMS: 257.1059 (MH⁺).

### e. 2-Methoxy-5-[(E)-2-(3,4,5-trimethoxyphenyl)vinyl]boronic acid (Trans-4)

Compound trans-4 was obtained in 76% yield (R_{f}=0.4, CHCl₂/MeOH=9/1) from the trans-3 following the same procedure as described above. ¹H NMR (CDCl₃, 400 MHz) δ 9.22 (s, 2H), 7.98 (s, 2H), 7.74 (d, 2H, J=8.0), 7.50 (d, 2H, J=8.0 Hz), 7.03 (dd, 2H, J=16.4, 16.4 Hz), 6.41 (m, 1H); ¹³C NMR (100 MHz) δ 157.14, 157.12, 138.60, 133.37, 128.67, 127.73, 124.93, 105.01, 102.16. Anal. Calcd for C₁₄H₁₃BO_{4.}0.5 H₂O: C, 63.38; H, 5.28. Found: C, 63.30, H, 5.07. HPLC: Method A, retention time=11.10 min; Method B, retention time=8.54 min; HRMS: 256.9723 (MH⁺).

### f. (E)-2-(4-(3,5-dimethoxystyryl)phenyl)-4,4,5,5-tetramethyl-1,3,2-dioxaborolane (7)

Substituted phosphonic acid diethyl esters (1.5 g, 5.3 mmol) in dry THF was added to NaH (0.25 g, 10.5 mmol) and 18-crown-6 (0.5 g, 2.1 mmol) in dry THF at 0° C. for 10 min 4-formylphenyl boronic acid pinacol ester (6.25 mmol) dissolved in dry THF were added drop wise to the above mixture at 0° C., and the mixture was stirred at room temperature for 1 h followed by heating to 60° C. for overnight. The mixture was quenched with cold water and extracted with EtOAc. The extracts were combined, washed with brine and dried over Na₂SO₄. The organic layer was filtered and rotary concentrated to give the crude mixture of trans stilbene 6. Flash column chromatography using 10% EtOAc/hexane eluted the trans-stilbene 6 (R_{f}=0.55, EtOAc/Hex=1/4) as a semi solid (0.83 g, 44%).

### g. (E)-4-(3,5-dimethoxystyryl)phenylboronic acid (trans-8)

KHF2 (0.4 g in 5 ml of methanol) was added to a solution of 7 (0.18 g, 0.5 mmol) in methanol (5 mL) and the mixture was stirred for 1 h. The white precipitate was filtered, washed with cold water and then washed with cold ether. The filtrate was dissolved in 10 ml of ethyl acetate (10 mL) and aqueous HCl (1N, 10 mL) and stiffing was continued for 1 h. The organic layer was separated and washed with a saturated solution of NaHCO3, brine, dried over Na2SO4. The organic layer was filtered and rotary concentrated to give the crude mixture of compound 3. Flash column chromatography using 30% EtOAc/hexane eluted the trans-8 (R_{f}=0.35, EtOAc/Hex=1/1) as a semi solid (0.04 g, 29%). HRMS ([M+H]) calcd, 284.1334. found, 284.1332.

¹H NMR (CD₃OD, 400 MHz) δ 7.73 (d, 1H), 7.56 (dd, 3H), 7.13 (s, 2H), 6.71 (d, 2H), 6.39 (s, 1H), 3.80 (s, 6H)

### h. 2-(3-iodo-5-methoxyphenyl)-4,4,5,5-tetramethyl-1,3,2-dioxaborolane (10)

Isopropyl borate (0.7 mL, 3.05 mmol) at −78° C. was added to a mixture of 3,5 di iodo anisole (7) (1 g, 2.77 mmol) in THF/Toluene (1:4) under nitrogen and stir for 30 min at −78° C. n-BuLi (1.6 M in hexane, 1.8 mL) was added drop wise and the mixture was stirred another 30 min at −78° C. The mixture is warm to −20° C. for 1 h while stirring. The reaction was quenched by HCl (1 N) and the mixture was stirred for 10 min. After neutralization with a saturated solution of aqueous NaHCO3, the mixture was extracted with ether, dried over anhydrous Na₂SO₄, filtered and then concentrated. The white solid obtained was dissolved in dichloromethane (15 mL) and pinacol (1.38 g, 11.7 mmol) was added. The reaction mixture was stirred for 3 h at room temperature, dried over anhydrous Na2SO4. The organic layer was filtered and rotary concentrated to give the crude mixture of compound 8. Flash column chromatography using 10% EtOAc/hexane eluted the 8 (R_{f}=0.51, EtOAc/Hex=1/4) as a semi solid (0.54 g, 54%).

¹H NMR (CHCl₃, 400 MHz) δ 7.69 (s, 1H), 7.32 (s, 1H), 7.25 (d, 1H), 3.77 (s, 3H), 1.32 (s, 12H).

### i. (E)-2-(3-methoxy-5-(4-methoxystyryl)phenyl)-4,4,5,5-tetramethyl-1,3,2-dioxaborolane (11)

To a stirred solution 1-methoxy-4-vinylbenzene (0.42 mL, 3.2 mmol) and compound 10 (1.4 g, 3.88 mmol) in DMF at room temperature under nitrogen were added benzyltriethylammonum chloride (0.73 g, 3.20 mmol), tributyl amine (1.97 mL, 8.30 mmol), and Palladium (II) acetate (0.033 g, 0.15 mmol). The resulting pale orange solution was stirred at 110° C. for overnight and allowed to cool to room temperature. The mixture was taken into dichloromethane and washed with water. The organic layer was separated and washed by water, 2N HCl (10 mL), water again, brine, dried over Na₂SO₄. The organic layer was filtered and rotary concentrated to give the crude mixture of compound 11. Flash column chromatography using 10% EtOAc/hexane eluted the 11 (R_{f}=0.37, EtOAc/Hex=1/4) as a semi solid (0.5 g, 43%).

¹H NMR (CDCl₃, 400 MHz) δ 7.56 (s, 1H), 7.43 (d, 2H), 7.22 (m, 1H), 7.11 (m, 2H), 6.92 (dd, 3H), 3.85 (s, 3H), 3.81 (s, 3H), 1.36 (s, 12H).

### j. (E)-3-hydroxy-5-(4-hydroxystyryl)phenylboronic acid (trans-12)

Compound 11 (0.1 g, 0.37 mmol) was dissolved in dry CH₂Cl₂ (10 ml) and cooled to −78° C., and BBr₃ (5 ml, 10 mmol, 1.0 M in CHCl₂) was added dropwise. The resulting mixture was stirred at −78° C. for another 1.5 h. The mixture was warmed to room temperature and stirred overnight. The reaction was quenched with H₂O (10 ml). Concentrated and applied to flash column chromatography using 10% CHCl₂/MeOH eluted the trans-12 (R_{f}=0.5, CHCl₂/MeOH=9/1) as a solid (0.04 g, 58%). HRMS ([M+H]) calcd, 256.1021. found, 256.1008).

### k. (E)-3-methoxy-5-(4-methoxystyryl)phenylboronic acid (trans-13)

KHF2 (0.42 g in 5 ml of methanol) was added to a solution of 11 (0.2 g, 0.5 mmol) in methanol (5 mL) and the mixture was stirred for 1 h. The white precipitate was filtered, washed with cold water and then washed with cold ether. The filtrate was dissolved in 10 ml of ethyl acetate (10 mL) and aqueous HCl (1N, 10 mL) and stiffing was continued for 1 h. The organic layer was separated and washed with a saturated solution of NaHCO3, brine, dried over Na2SO4. The organic layer was filtered and rotary concentrated to give the crude mixture of compound 5. Flash column chromatography using 10% CHCl₂/MeOH eluted the compound 4 (R_{f}=0.5, CHCl₂/MeOH=19/1) as a solid (0.08 g, 52%). HRMS ([M+H]) calcd, 284.1334. found, 284.1246).

¹H NMR (CD₃OD, 400 MHz) δ 7.47 (m, 3H), 7.09 (m, 2H), 7.00 (d, 2H), 6.89 (d, 2H), 3.82 (s, 3H), 3.79 (s, 3H)

### 4. Results

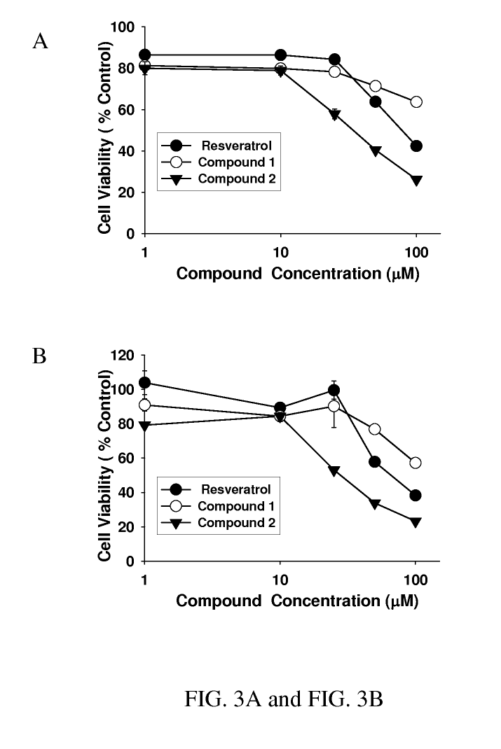

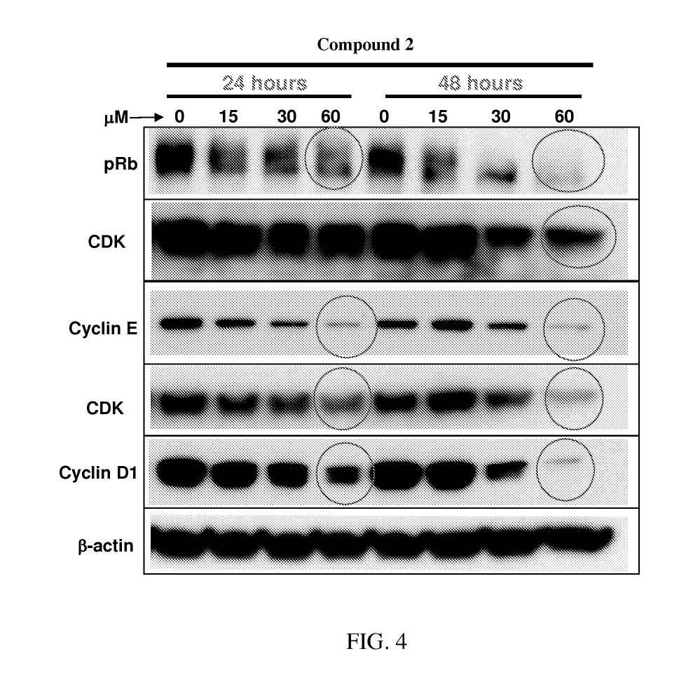

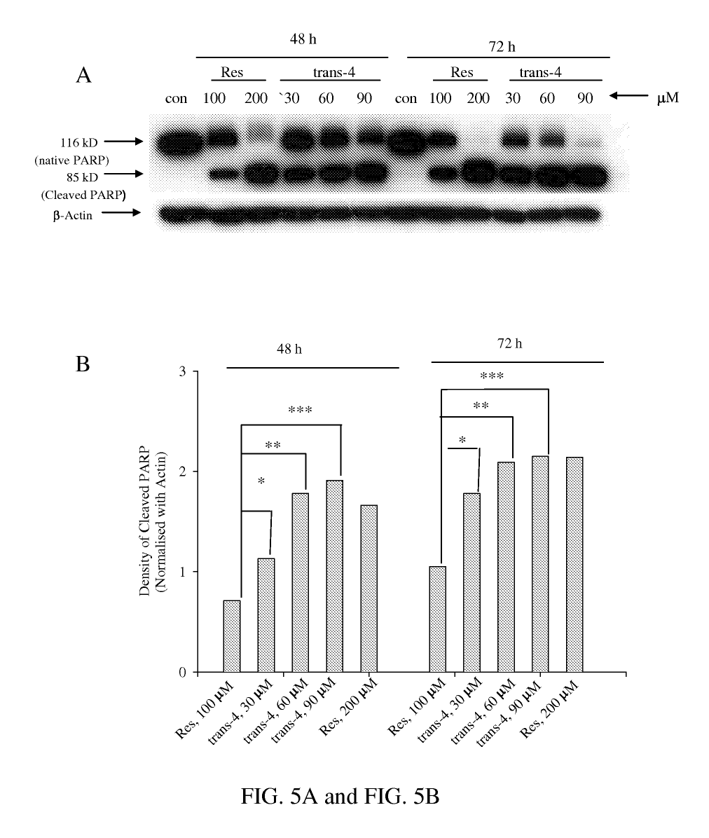

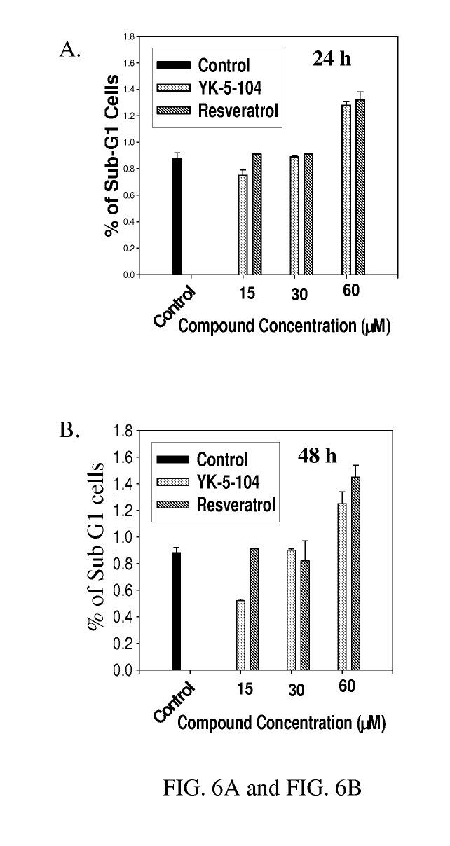

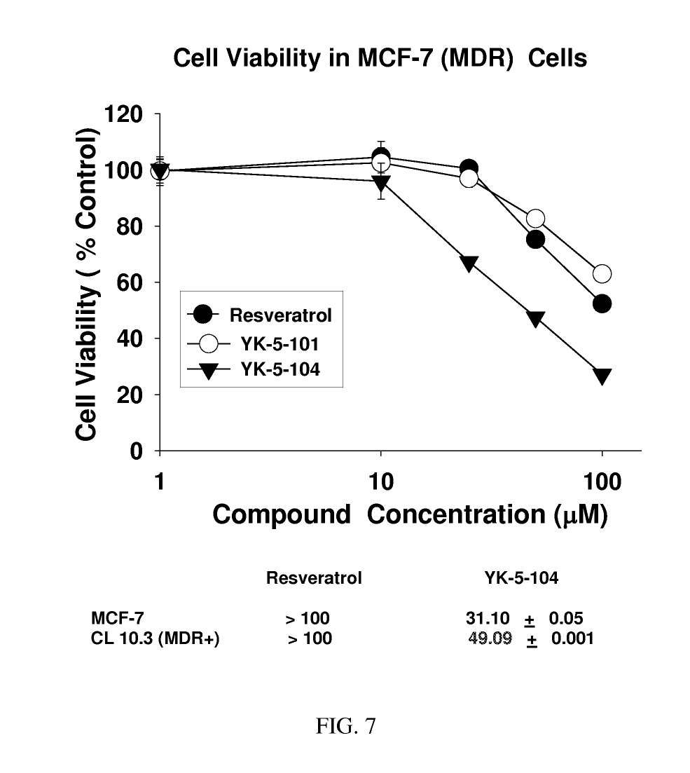

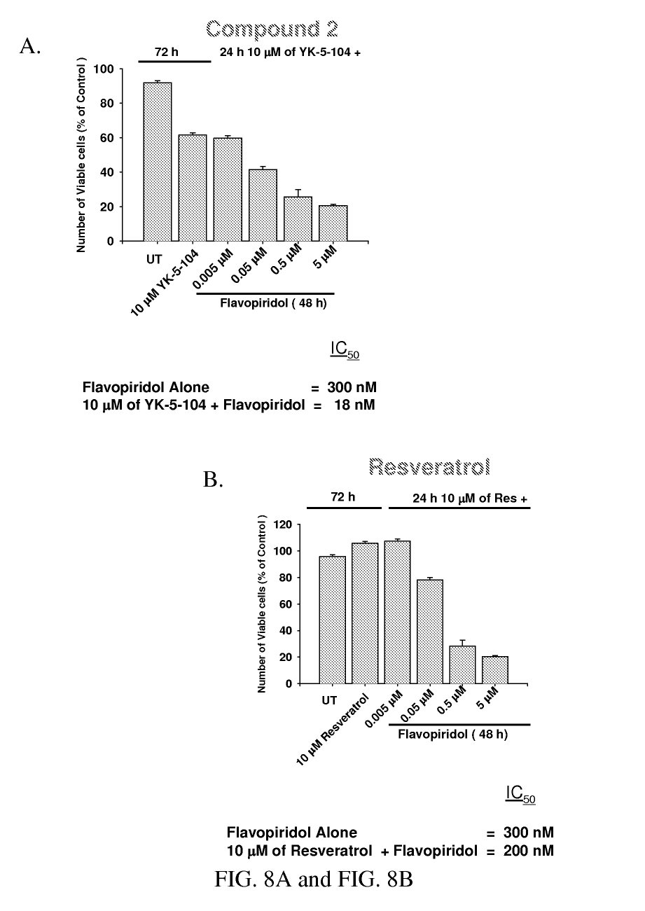

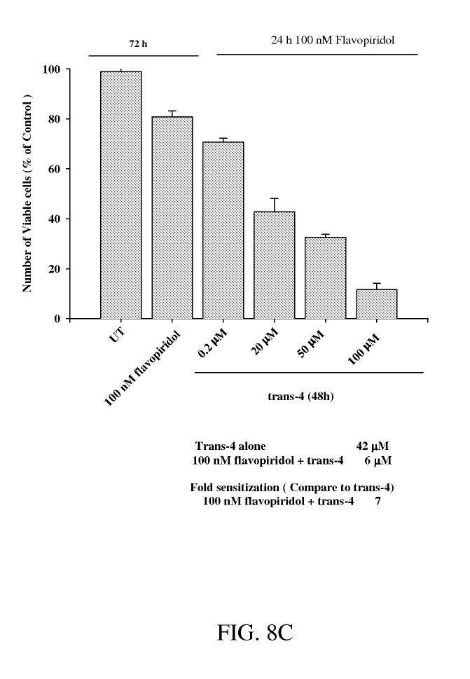

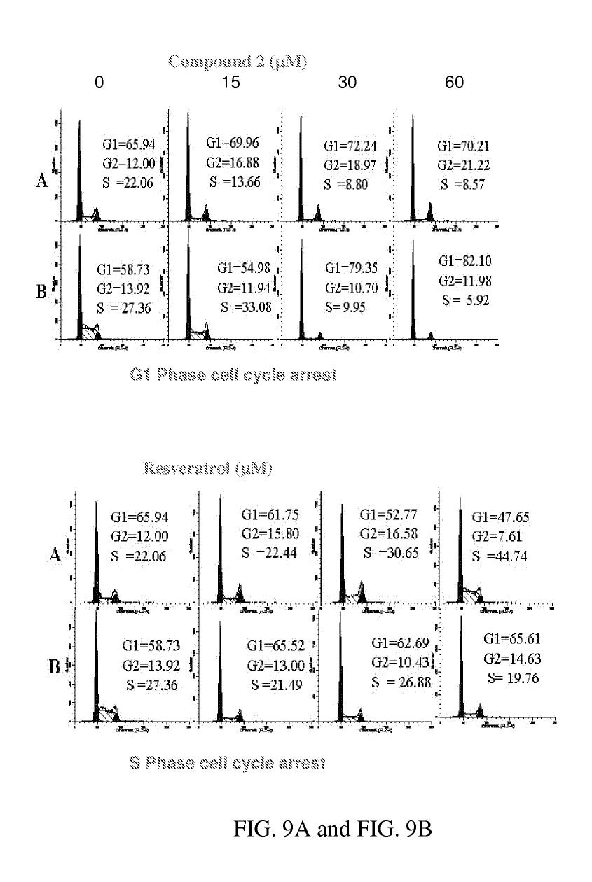

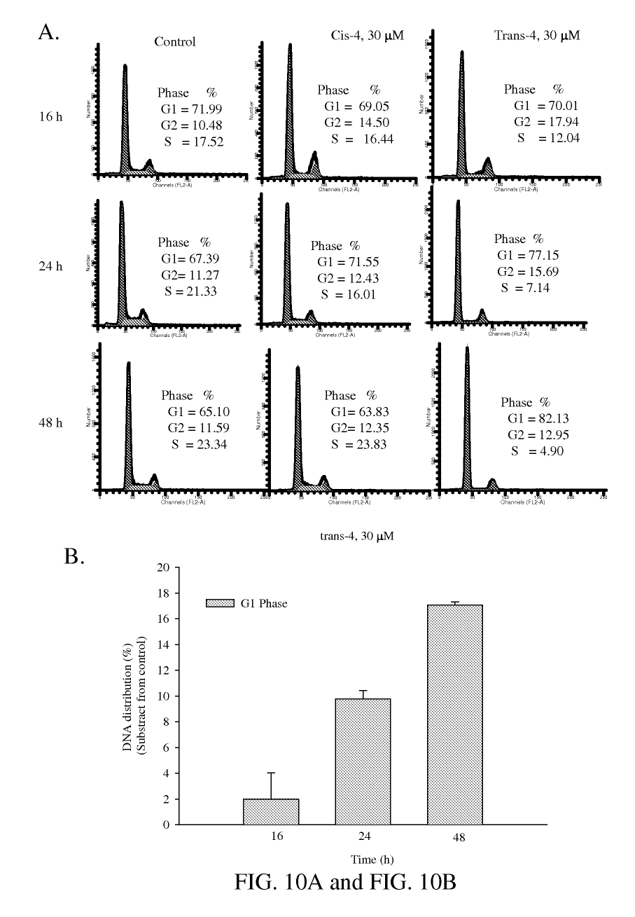

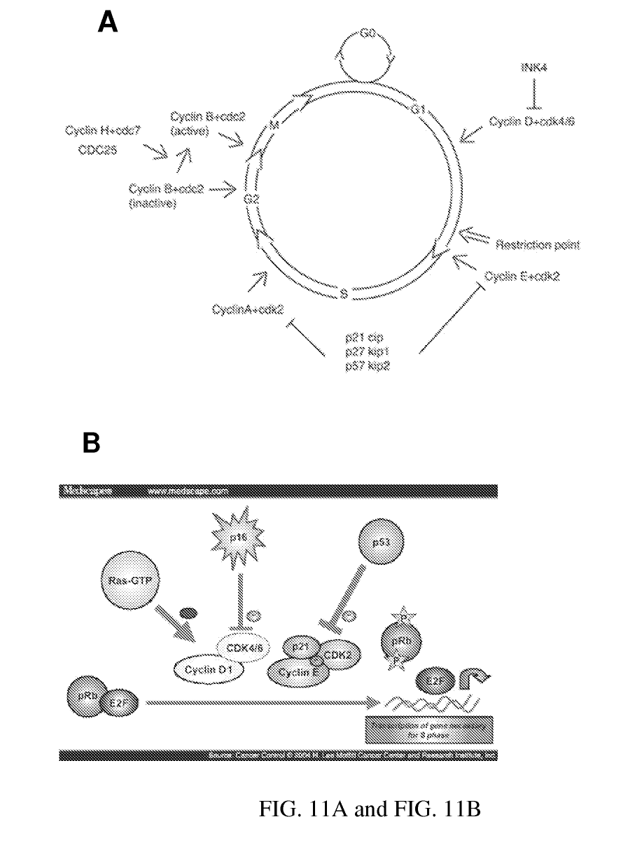

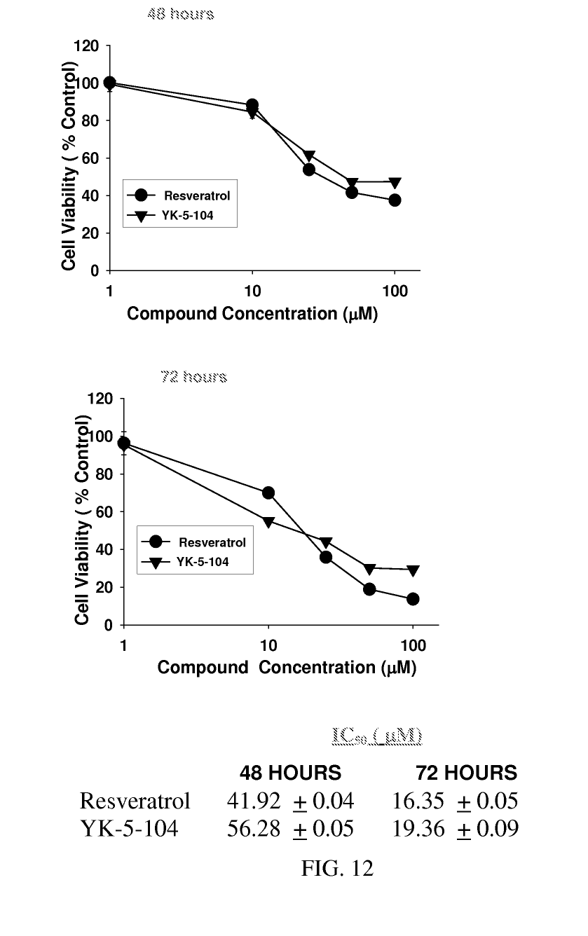

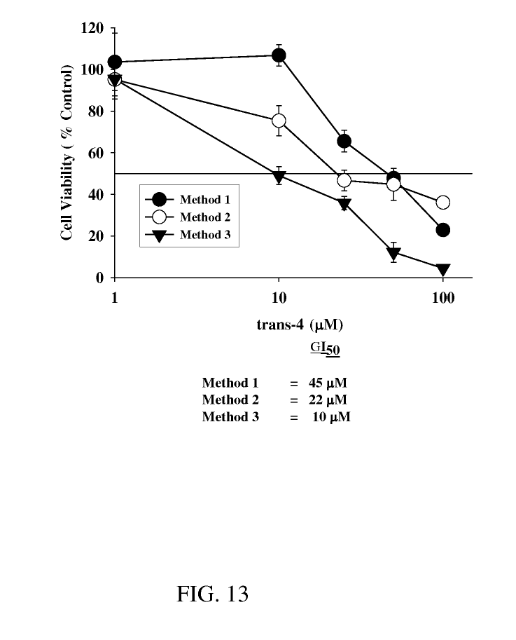

Disclosed is a class of potent boronic acid derivatives of resveratrol (FIG. 2 and Scheme 1). These disclosed compounds demonstrated improved cellular toxicity against estrogen dependant human breast cancer cells as compared to RSV (FIG. 3). Treatment with YK-5-104 induced G1 arrest in estrogen dependant human breast cancer cells (FIG. 10). The inhibition of the expression level of pRb and positive regulators of Rb (CDK's) in MCF-7 cells, further supports that YK-5-104 decreases the pRb levels, which halts cell division at the G1 Phase (FIG. 4). Mechanistic studies revealed an increase in the level of the hypodiploid population (Sub-G1) and PARP cleavage in MCF-7 cells supports that YK-5-104 potentiates apoptosis (FIGS. 5 and 6). YK-5-104 exhibits an irreversible antiproliferative effect, which can provide a therapeutic advantage. (FIG. 13). Interestingly, YK-5-104 showed similar activity in a multidrug resistance cell line (FIG. 7) and it is not a substrate for p-glycoprotein. YK-5-104 also modulates the flavopiridol mediated cell viability, which suggest that the present compound may be used for combination therapy with a CDK inhibitor (FIG. 8). Finally, the potent irreversible anti-proliferative and apoptotic effects of the present boronic acid derivative of RSV provides a platform for advancing this compound into preclinical studies.

i. Inhibition of MCF-7 Human Breast Cancer Cell Growth

To examine the anti-proliferative effect of newly synthesized cis-4 and trans-4, the effects of cis-4 and trans-4 were first compared to that of resveratrol on the growth of ER+, estrogen-dependent MCF-7 human breast cancer cells using the WST-1 assay as described in materials and methods. As shown in Table 1, the trans-4 analog is cytotoxic towards MCF-7 cell growth in a time and concentration-dependent manner, with a growth-inhibitory 50 (GI₅₀) value in MCF-7 cells of 36.6 μM±0.06. In contrast, the GI₅₀ is >100 μM for trans-resveratrol at 48 h and the cis-4 analog does not show any growth inhibition at 100 μM. Identical results were obtained at 72 hours of treatment (Table 1). These data show that trans-4 is a more potent inhibitor of MCF-7 cell growth compared to resveratrol and this warrants further examination of its mechanism of action.

TABLE 1
Effects of cis-4, trans-4 or resveratrol on the survival of the
ER+, estrogen-dependent human breast cancer MCF-7 cell line.

Cell Survival
(WST-1 assay, GI₅₀, μM)

| Compound | 48 hours | 72 hours |
|---|---|---|
| trans-4 | 36.6 ± 0.06 | 31.1 ± 0.05 |
| cis-4 | >100 | >100 |
| Resveratrol | >100 | >100 |

Cis-4, Trans-4 and resveratrol were tested at various concentrations for effects on cell survival of breast cancer cells.
Cell survival was estimated 48 h and 72 h after the addition of each compound using the WST-1 reduction assay.
Results shown are mean values of triplicate experiments.
The GI₅₀ value (the concentration yielding 50% growth inhibition) was interpolated from FIG. 3 showing the graph of the log of compound concentration versus the fraction of surviving cells.
The GI₅₀ was calculated using Graph Pad Prism.
Data are expressed as a mean (±SEM) of triplicate experiments?

ii. Growth Inhibitory Effects of Trans-4 in a Multidrug Resistant Variant of MCF-7 Cells

During chemotherapy, breast tumor cells may either develop resistance to a single drug or combination of drugs that share similar mechanisms, or display cross-resistance to functionally and structurally unrelated drugs (Curr Opin Oncol, 12, 450, 2000). This phenomenon is known as multidrug resistance (MDR). Over-expression of P glycoprotein 170 (Pgp-170, the product of the MDR1 gene) is one of the most common causes of MDR and is expressed in many types of cancer cells including breast and ovary (Int J Clin Pharmacol Ther, 36, 29, 1998). Therefore, whether trans-4 could effectively inhibit the growth of CL10.3 cells, a derivative of MCF-7 that over-expresses the MDR gene (J. Natl. Cancer Inst, 84, 1506, 1992) needed to be determined. As shown in Table 2, trans-4 is equipotent in CL 10.3 and MCF-7 cells. This is in contrast to paclitaxel, a known substrate of MDR1, (Breast Cancer Res Treat, 33, 27, 1995) which strongly inhibits MCF-7 cell growth but has no effect on CL 10.3 cells. These results clearly demonstrate that compound 2 is effective in multidrug resistant breast cancer cells and therefore can have potential therapeutic utility.

TABLE 2
Effects of trans-4 or paclitaxel on the survival of multidrug
resistant breast cancer cell lines.
Cell Survival
(WST-1 assay, GI₅₀, μM, 72 h)

| Cell line | Origin | trans-4 | Paclitaxel |
|---|---|---|---|
| MCF-7 | Breast | 31.10 ± 0.05 | 0.002 ± 1.12 |
| CL 10.3 | Breast (MDR) | 49.09 ± 0.001 | >50 |
| MDR GI50/ | | 1.57 | >25,000 |
| non-MDR GI50 | | | |

trans-4 or Paclitaxel were tested at various concentrations for effects on cell survival of breast cancer cells.
Cell survival was estimated 72 h after the addition of each compound using the WST-1 reduction assay.
Results shown are mean values of triplicate experiments.
The GI₅₀ value (the concentration yielding 50% growth inhibition) was interpolated from the graph of the log of compound concentration versus the fraction of surviving cells.
The GI₅₀ was calculated using Graph Pad.
Data are expressed as mean (±SEM) of triplicate experiments

iii. Effect of Trans-4 on Cell Cycle Distribution in MCF-7 Cells

To determine whether the growth inhibitory effect of trans-4 occurs through a blockade of cell cycle progression, the DNA content was analyzed in each phase of the cell cycle by flow cytometry. MCF-7 cells treated with DMSO (vehicle control), cis-4 and trans-4 for 16, 24, or 48 hours were subjected to flow cytometric analysis. As shown in FIG. 10A, trans-4 induced a time-dependent accumulation of cells in the G1 compartment relative to vehicle control. The G1 phase accumulation was paralleled by a marked reduction in the percentage of cells in S phase. Trans-4 at 30 μM reached the highest level of G1 arrest by a 17% in G1 cells compared to control at 48 hours (FIG. 10B) indicate that trans-4 blocks MCF-7 cell cycle progression in the G1 phase, which can contribute to its cytotoxic effects.

iv. Irreversible Effect of Trans-4 on MCF-7 Cell Growth Inhibition

Because trans-4 was a more potent inhibitor of MCF-7 cell growth compared to resveratrol, whether the growth inhibition is irreversible or reversible was examined. Towards this end, cells were treated with trans-4 under three different conditions as described above in the materials and methods using the WST-1 assay. As shown in FIG. 13, the GI₅₀ for trans-4 after 48 h treatment is 45 μM (Method 1). Treatment of cells with trans-4 for 48 h followed by recovery in fresh media without trans-4 for an additional 48 h (Method 2) gave a GI₅₀ of 22 μM which is two-fold lower than Method 1. These data indicate that the anti-proliferative effect of trans-4 in MCF-7 cells is irreversible. Treatment of cells with trans-4 for 72 h with media changes (containing trans-4) after every 24 h (Method 3) gave a GI₅₀ of 10 μM. This result indicates that sequential dosing with trans-4 enhances its effect on growth inhibition, which is important from a therapeutic treatment point of view. The irreversible anti-proliferative effect of compound 2 was further confirmed by flow cytometry

v. Effect of Trans-4 on Expression Level of G1 Cell Cycle Proteins in MCF-7 Cells

Whether the cell cycle arrest in the G1 phase induced by trans-4 was related to the expression of G1 cell cycle positive regulatory proteins that regulate the G1-to-S transition was investigated; these include cyclin D1 and cyclin E, their associated cyclin-dependent kinases (cdk4, cdk2), and the phosphorylation state of pRb. MCF-7 cells were treated with the indicated concentrations of trans-4 for 24 h and 48 h, then harvested for immunoblotting. As shown in FIG. 4, compound 2 decreases the expression level of cdk4, cdk2, cyclin E, cyclin D1 and pRb, which are responsible for cell cycle progression early in the G1 phase, with the greatest effect observed at 48 hours exposure. This down-regulation of G1-S positive regulatory proteins correlates with the observed accumulation of cells in the G1 phase in trans-4 treated MCF-7 cells.

vi. Apoptotic Changes in MCF-7 Cells in Response to Trans-4

To characterize whether trans-4 could induce apoptotic cell death in MCF-7 cells, cleavage of the caspase substrate PARP was examined by immunoblotting. As shown in FIGS. 5A and 5B, cells treated with 30 μM compound 2 showed robust expression of the 85-KDa cleavage fragment of PARP. This band is more intense in trans-4 treated MCF7 cells compare to resveratrol, which included similar levels of PARP cleavage at 200 μM. These results demonstrate that the trans-4 stronger anti-proliferative effect is also associated with a more potent induction of apoptosis when compared to resveratrol.

vii. Effect of Trans-4 on E2-Mediated Stimulation of MCF-7 Cell Growth.

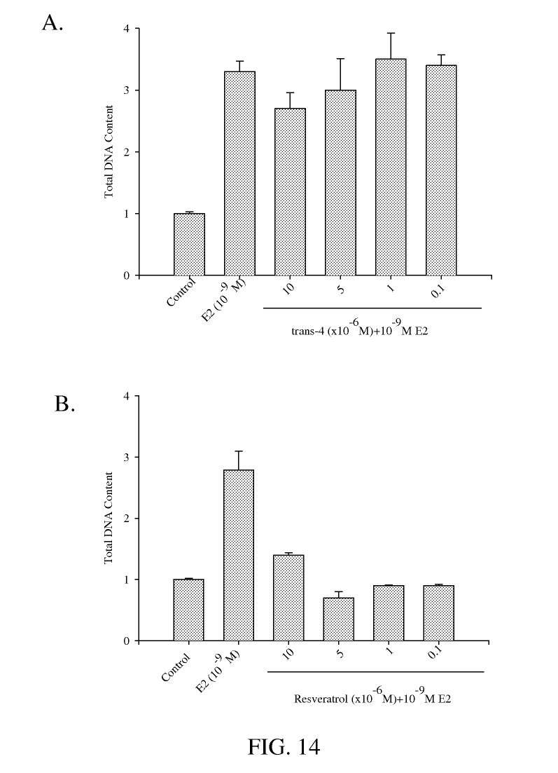

The 4′ hydroxyl group of resveratrol mimics the steroid estrogen (Estradiol, E2), which activates ER and stimulates down-stream signaling pathways in estrogen-dependent breast cancer cell lines ((Proc Natl Acad Sci U.S A 94, 14138; 1997, Int J Cancer 104, 587: 2003) However, resveratrol can have a mixed agonist/antagonist activity towards E2-mediated MCF-7 cell growth. Therefore the effect of trans-4 on E2-mediated cell growth was examined to check trans-4 action against ER and to determine whether it acts as an agonist or antagonist. MCF-7 cells were seeded in a phenol-red free cell culture media and after 24 hours, cells were treated with the indicated concentrations of resveratrol or trans-4 in the presence of a constant E2 concentration (10⁻⁹M). As shown in FIG. 14A, trans-4 does not have any effect on E2-induced cell growth and therefore appears to have neither agonist nor antagonist activity toward ER activity in MCF-7 cells. In contrast, resveratrol antagonizes E2-mediated MCF-7 cell growth (FIG. 14B). These results convincingly demonstrate that, due to structural alteration, trans-4 utilizes a different mechanism to inhibit MCF-7 cell growth which is independent of the estrogen receptor.

viii. Effect of Trans-4 in Combination with G1 Phase Selective Drugs

In general chemotherapy was applied in combination with drugs with the same or different cellular mechanism compared to the single drug in order to increase efficacy of the drugs and concomitantly reduce the undesired side effects exerted by cytostatic drugs. In view of this, the effect of trans-4 was examined in combination with flavopirdol. A known compound undergoing G1 phase arrest in human breast cancer cell lines was studied. In the continuation, cells were treated in two administrative methods to see the effect of trans-4 in combination treatment. As shown in the FIG. 8A, MCF-7 cells treated with flavopiridol only had a GI₅₀ value of 300 nM. where as in combination of trans-4 with flavopiridol they had a GI₅₀ value of 18 nM respectively. Thus trans-4 modulates the flavopiridol mediated MCF-7 cell growth inhibition by a 16.6 fold increase compared with flavopiridol alone. As shown in FIG. 8C, the opposite administration of the compounds resulted in a 7 fold increase in growth inhibition of MCF-7 cell as compared with trans-4. Resveratrol does not sensitize the MCF-7 cells incubated with flavopiridol (FIG. 8B). These results indicated that trans-4 can be useful in combination of different agents in chemotherapy.

### 5. Conclusions

Resveratrol, a naturally occurring phytoalexin present in the skin of red grapes and other medicinal plants, has received broad attention due to its potential as a chemotherapeutic and cancer prevention agent. Previously resveratrol has been shown to have growth inhibitory effects in different human cancer cell lines of both hematological and epithelial origin including breast, colorectal, leukemia, and epidermoid carcinoma. However the effects of resveratrol in breast cancer cell growth inhibition are not consistent. At high doses, resveratrol act as a growth inhibitor while at lower doses RSV stimulates growth in ER+ and ER− breast cancer cells (Life Sci 66, 769, 2000). In the presence of estrogen (E2), resveratrol shows agonistic as well as antagonistic actions on the growth of ER+ cells. Previously it was shown that a set of CA-4 and chalcone derivatives of CA-4 containing a boronic acid modification have potent antiproliferative effects. (Chem Biol, 12, 1007. 2005; Bioorg. Med. Chem., 18, 971, 2010). In addition, the boronic acid moiety shows activity in several pharmaceutical agents such as enzyme inhibitors, ((Mol. Cancer. Ther. 8, 3234, 2009; Cancer Res., 70, 1970, 2010) carbohydrate recognizing sensors (Curr. Org. chem., 6, 1285, 2002) and boron neutron capture therapy ((Chem. Rev, 98, 1515, 1998). In the present study, the boronic acid mimics of resveratrol were designed and synthesized by chemically modifying the 4′-OH to boronic acid leading to development of analogs herein, including two analogs as shown in FIG. 2 and Scheme 1. As shown in Table 1, trans-4 has the greatest activity in inhibiting MCF-7 cell growth compared to resveratrol. Previous studies of MCF-7 cell growth inhibition by resveratrol is accompanied by S-phase cell cycle arrest at much higher doses (300 μM) (Clin Cancer Res 2002, 8, 893-903). This also raises the possibility that growth inhibition by trans-4 might also be attributed to cell cycle disruption. It was observed that trans-4 induced a G1 phase cell cycle arrest at 30 μM in a time dependent manner as shown in FIG. 10. This different mechanism (G1 vs. S phase arrest) might be the underlying cause for the more potent cell inhibitory induced by trans-4 as compared to resveratrol. The G1/S transition is a key regulatory point where a cell decides whether or not to enter into DNA replication (S phase) (FEBS Letters, 1999, 458, 349-353). Hence, molecules that inhibit the G1/S phase transition are often considered to have excellent therapeutic significance. Importantly, trans-4 also showed an irreversible growth inhibitory effect (FIG. 13).

The growth inhibitory action of trans-4 was also assessed in multidrug resistant (MDR) human breast cancer cells (CL 10.3, derived from MCF-7 breast cancer cells transfected with the human mdr 1 gene). These data show that trans-4 still has significant activity in the MDR cell line and is therefore likely not a substrate for P-glycoprotein. Inhibition of the expression level of G1 phase regulatory proteins (cyclins D1 and E, CDK2 and CDK4, pRb) in MCF-7 cells (FIG. 4), further indicates that trans-4 acts by halting cell division at the G1/S check point. Treatment of cancer cells with the trans-4 also strongly induces apoptosis as measured by the 85 KD cleaved PARP fragment, and it did so at a much lower concentration than resveratrol (FIG. 5). Finally, it appears that the molecular mechanism of compound 2 action does not involve the estrogen receptor or the inhibition of E2-induced cell growth (FIG. 14).

In summary, a new class of boronic acid biomimetics of resveratrol have been designed and synthesized that are potent growth inhibitors with G1 cell cycle arrest and pro-apoptotic capabilities in MCF-7 cells. Furthermore, trans-4 is also shown to be an irreversible growth inhibitor in MCF-7 cells and have a potent effect in multidrug resistant cells.

1. A compound, or a pharmaceutically acceptable salt, prodrug, clathrate, tautomer or solvate thereof, wherein the compound has the structure: 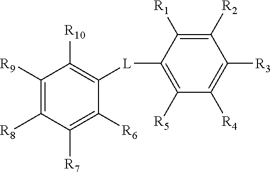
wherein
R¹, R², R³, R⁴, R⁵, R⁶, R⁷, R⁸, R⁹ and R¹⁰ are independently hydrogen, —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino;
R⁸ and R⁹ are optionally cyclized to form cylcoalkyl, aryl, heteroaryl or heterocyclyl, optionally substituted with —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino;
L is 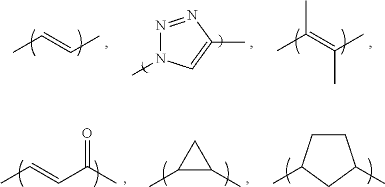
or L is absent when R⁸ and R⁹ are cyclized;
wherein at least one position in the compound is substituted with —B(OH)₂, and at least one position in the compound is substituted with alkoxy, alkoxydialkylamino or hydroxyl; and
wherein the compound is not

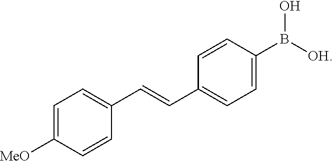

2-3. (canceled)

4. The compound of claim 1, wherein R³ is —B(OH)₂, hydroxyl or C₁-C₃ alkoxy.

5-6. (canceled)

7. The compound of claim 1, wherein L is present and is: 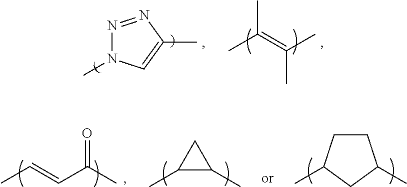
or L is absent when R⁸ and R⁹ are cyclized.

8. (canceled)

9. The compound of claim 1, having the structure 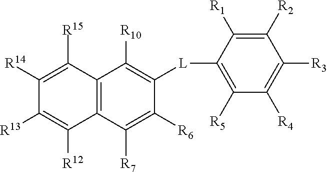
wherein:
R¹², R¹³, R¹⁴ and R¹⁵ are independently hydrogen, —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino or sugars.

10. (canceled)

11. The compound of 1, having the structure:

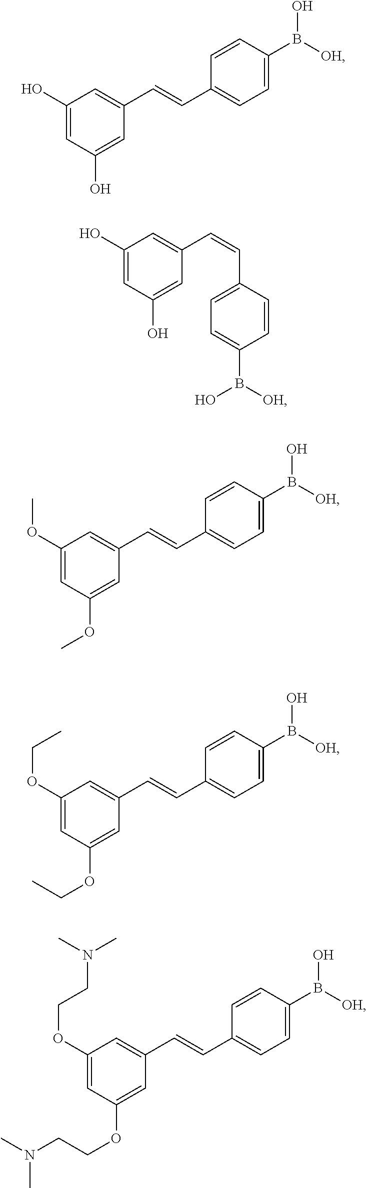

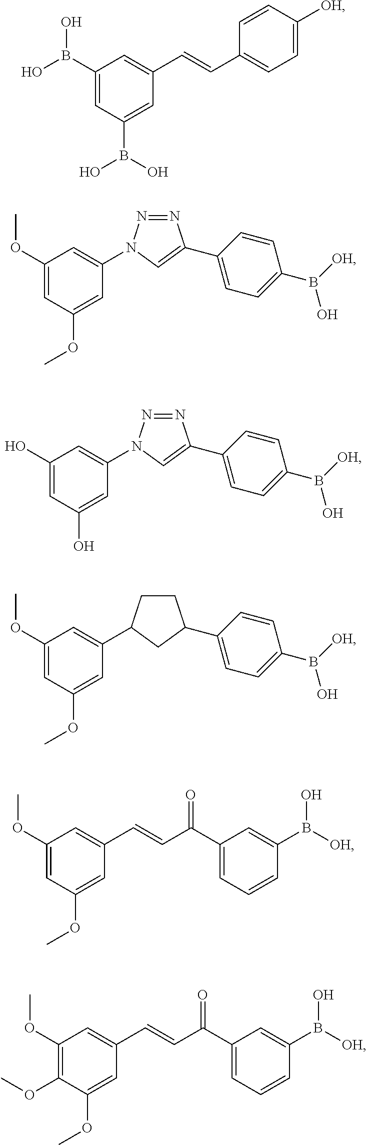

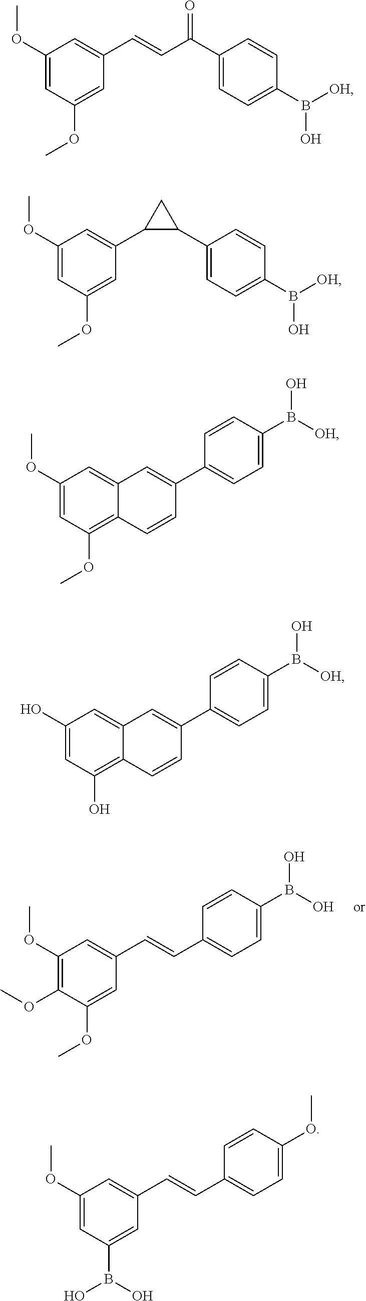

12. (canceled)

13. A composition comprising, a compound, of structure A-L-C, or a pharmaceutically acceptable salt, prodrug, clathrate, tautomer or solvate thereof, wherein:
A is substituted or unsubstituted cylcoalkyl, aryl, heteroaryl, heterocyclyl;
L is present or absent, if present L is C₁-C₆ alkyl, C₂-C₆ alkenyl, aryl, heteroaryl, cycloalkyl, heterocyclyl, —P-Q-S—, wherein P is C₁-C₆ alkyl, C₂-C₆ alkenyl, aryl, heteroaryl, cycloalkyl or heterocyclyl, Q is —N(R¹¹)—, —O—, —S—, —C(O)—, wherein R¹¹ is hydrogen or C₁-C₃ alkyl, S is present or absent, if present S is C₁-C₆ alkyl, C₂-C₆ alkenyl, aryl, heteroaryl, cycloalkyl or heterocyclyl; and
C is substituted or unsubstituted cylcoalkyl, aryl, heteroaryl, heterocyclyl, wherein at least one position in the compound is substituted with —B(OH)₂, and at least one position in the compound is substituted with alkoxy, alkoxydialkylamino or hydroxyl.

14. The composition of claim 13, wherein the structure A-L-C has the structure 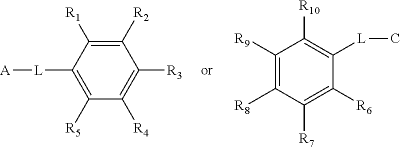
wherein:
R¹, R², R³, R⁴, R⁵, R⁶, R⁷, R⁸, R⁹ and R¹⁰ are independently hydrogen, —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino; and
R⁸ and R⁹ are optionally cyclized to form cylcoalkyl, aryl, heteroaryl or heterocyclyl, optionally substituted with —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino.

15. The composition of claim 14, having the structure

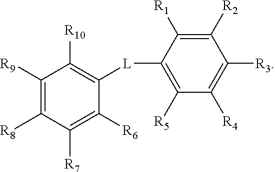

16. The composition of claim 15, wherein R³ is —B(OH)₂, hydroxyl or C₁-C₃ alkoxy.

17-18. (canceled)

19. The composition of claim 15, wherein L is present and is:

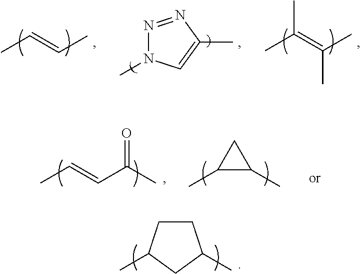

20. The composition of claim 15, wherein L is absent.

21. The composition of claim 15, having the structure 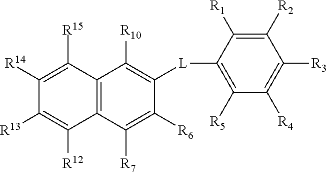
wherein:
R¹², R¹³, R¹⁴ and R¹⁵ are independently hydrogen, —B(OH)₂, alkyl, alkenyl, alkynyl, halo, alkoxy, amino, alkylamino, dialkylamino, cyano, nitro, formyl, carboxyl, alkoxycarbonyl, aminocarbonyl, alkoxydialkylamino, alkylaminocarbonyl, dialkylamino carbonyl, haloalkyl, haloalkloxy, haloalkylamino, or di(haloalkyl)amino.

22. The composition of claim 15 wherein, R¹³ and R¹⁵ are hydrogen and R¹² and R¹⁴ are independently —B(OH)₂, hydroxyl, C₁-C₃ alkoxy or C₁-C₃ alkoxydialkylamino.

23. The composition of claim 15, having the structure:

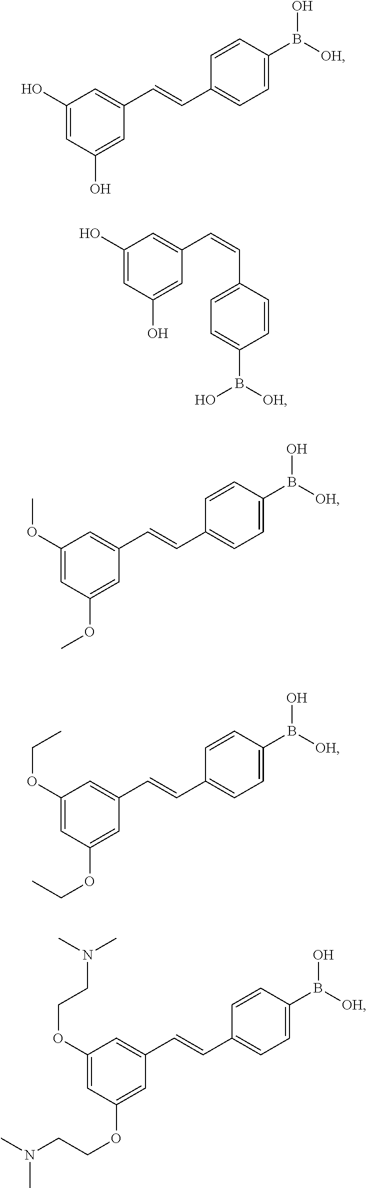

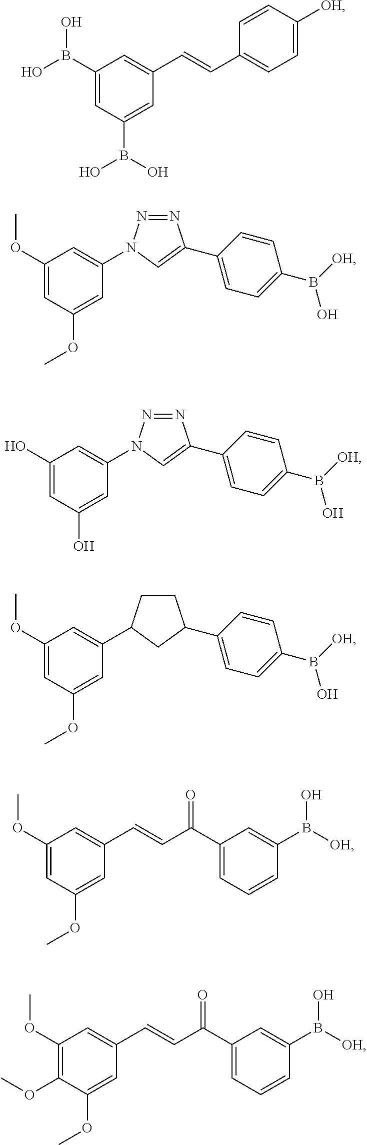

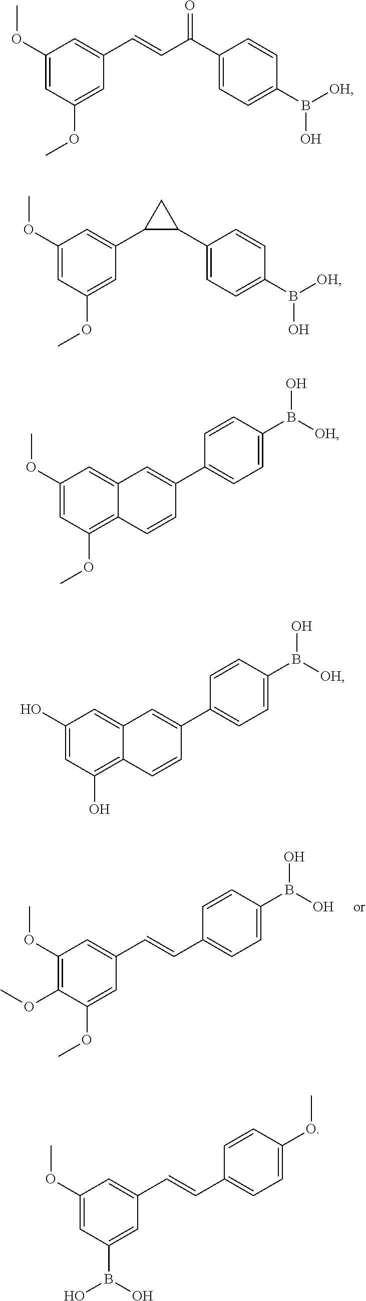

24. (canceled)

25. A method of treating cancer comprising, administering to a subject in need of treatment a composition comprising, a compound of structure A-L-C, or a pharmaceutically acceptable salt, prodrug, clathrate, tautomer or solvate thereof, wherein:
A is substituted or unsubstituted cylcoalkyl, aryl, heteroaryl, heterocyclyl;
L is present or absent, if present L is C₁-C₆ alkyl, C₂-C₆ alkenyl, aryl, heteroaryl, cycloalkyl, heterocyclyl, —P-Q-S—, wherein P is C₁-C₆ alkyl, C₂-C₆ alkenyl, aryl, heteroaryl, cycloalkyl or heterocyclyl, Q is —N(R¹¹)—, —O—, —S—, —C(O)—, wherein R¹¹ is hydrogen or C₁-C₃ alkyl, S is present or absent, if present S is C₁-C₆ alkyl, C₂-C₆ alkenyl, aryl, heteroaryl, cycloalkyl or heterocyclyl; and
C is substituted or unsubstituted cylcoalkyl, aryl, heteroaryl, heterocyclyl, wherein at least one position in the compound is substituted with —B(OH)₂, and at least one position in the compound is substituted with alkoxy, alkoxydialkylamino, or hydroxyl.

26. The method of claim 25, wherein the structure A-L-C has the structure 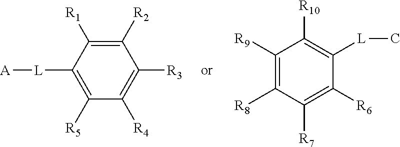
wherein:
R¹, R², R³, R⁴, R⁵, R⁶, R⁷, R⁸, R⁹ and R¹⁰ are independently hydrogen, —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino; and
R⁸ and R⁹ are optionally cyclized to form cylcoalkyl, aryl, heteroaryl or heterocyclyl, optionally substituted with —B(OH)₂, mild lewis acid, strong acid, weak acid, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, di(haloalkyl)amino or sugars.

27. The method of claim 26, having the structure

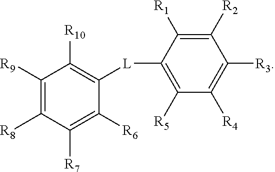

28. The method of claim 27, wherein R³ is —B(OH)₂, hydroxyl or C₁-C₃ alkoxy.

29-30. (canceled)

31. The method of claim 27, wherein L is present and is:

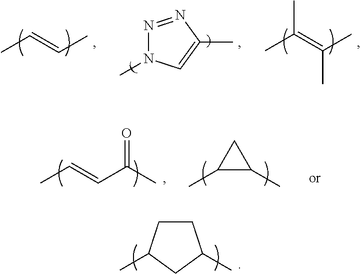

32. The method of claim 27, wherein L is absent.

33. The method of claim 27, having the structure 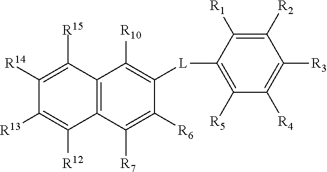
wherein:
R¹², R¹³, R¹⁴ and R¹⁵ are independently hydrogen, —B(OH)₂, alkyl, alkenyl, alkynyl, halo, alkoxy, amino, alkylamino, dialkylamino, cyano, nitro, formyl, carboxyl, alkoxycarbonyl, alkoxydialkylamino, aminocarbonyl, alkylaminocarbonyl, dialkylamino carbonyl, haloalkyl, haloalkloxy, haloalkylamino, or di(haloalkyl)amino.

34. The method of claim 33 wherein, R¹³ and R¹⁵ are hydrogen and R¹² and R¹⁴ are independently —B(OH)₂, hydroxyl, C₁-C₃ alkoxy or C₁-C₃ alkoxydialkylamino.

35. The method of claim 27, having the structure:

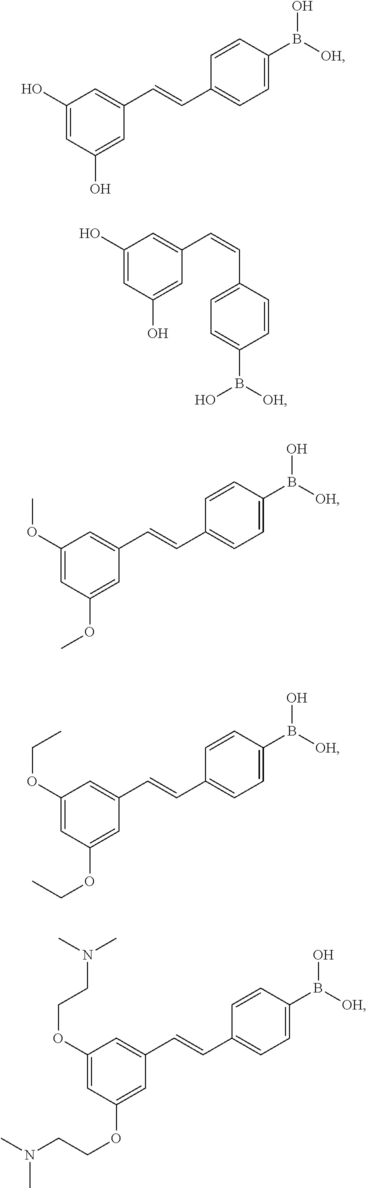

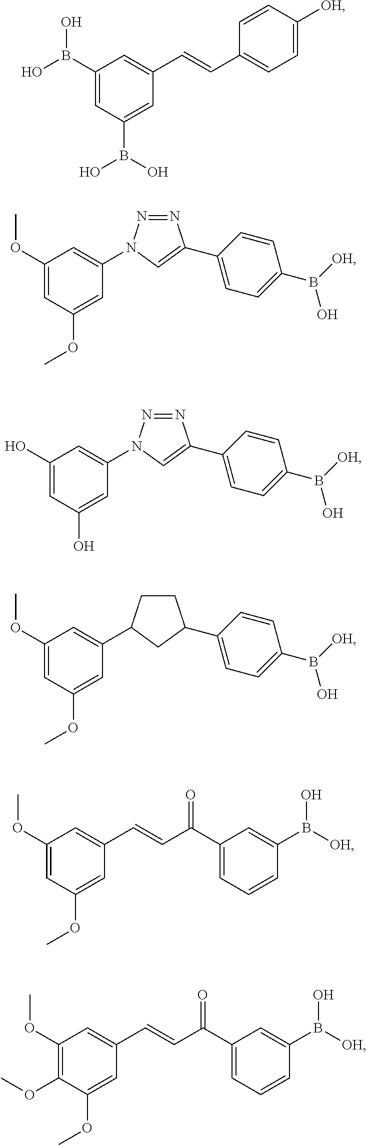

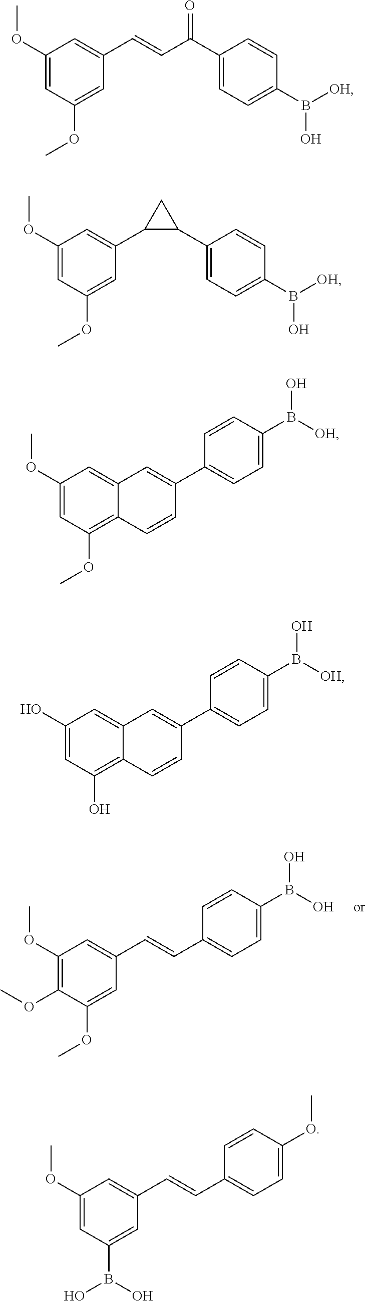

36. (canceled)

37. The method of claim 25, wherein the subject has been assayed for cancer or a risk of cancer.

38. The method of claim 25, wherein the subject is at risk of having cancer.

39. The method of claim 25, wherein the subject is diagnosed with cancer.

40. The method of claim 25, wherein the cancer express ER.

41. The method of claim 25, wherein the cancer is breast cancer.
